(19) United States
(12) Patent Application Publication
(10) Pub. No.: US 2026/0286304 A1
(43) Pub. Date: Sep. 24, 2026

(54) CHIMERIC ANTIGEN RECEPTORS AND METHODS OF USE THEREOF

(71) Applicant: Legend Biotech USA Inc., Somerset, NJ (US)

(72) Inventors: Fengyuan TANG, Nanjing (CN); Wang ZHANG, Nanjing (CN); Qiuchuan ZHUANG, Nanjing (CN); Rui GAO, Nanjing (CN); Qianqian LIU, Nanjing (CN); Xiao YANG, Nanjing (CN)

(21) Appl. No.: 19/478,148

(22) PCT Filed: May 10, 2024
(86) PCT No.: PCT/CN2024/092129

§ 371 Date: Oct. 23, 2025

(30) Foreign Application Priority Data
May 10, 2023 (WO) PCT/CN2023/093263

(51) Int. Cl. C12N 5/0783; A61K 40/11; A61K 40/31; A61K 40/42; A61P 35/00; C07K 16/28; C12N 15/86

(52) U.S. Cl. CPC C12N 5/0636; A61K 40/11; A61K 40/31; A61K 40/4202; A61K 40/421; A61P 35/00; C07K 16/28; C07K 16/2803; C12N 15/86; C07K 2317/31; C07K 2317/53; C07K 2317/565; C07K 2317/569; C07K 2317/622; C07K 2319/02; C07K 2319/03; C12N 2510/00; C12N 2740/15043

(57) ABSTRACT

Disclosed is a dual chimeric antigen receptor (Dual-CAR) system targeting neuroendocrine lineage markers, comprising (1) a first CAR comprising a primary intracellular signaling domain of an immune cell and (2) a second CAR that does not comprise a primary intracellular signaling domain derived from CD3ζ or a primary intracellular signaling domain of an immune cell. Relates to a single CAR system targeting a neuroendocrine lineage marker. Also described are engineered immune cells comprising such systems and the use of these cells in the treatment of cancer.

### TECHNICAL FIELD

This application claims benefit of priority of International Patent Application No. PCT/CN2023/093263 filed on May 10, 2023, the content of which is incorporated herein by reference in its entirety.

### SEQUENCE LISTING

This application incorporates by reference a Sequence Listing submitted with this application as XML file format, entitled “IEC240077PCT SEQUENCE LISTING.xml,” created on May 7, 2024 having a size of 61,750 bytes.

### TECHNICAL FIELD

This disclosure relates to chimeric antigen receptors (CARs), a dual chimeric antigen receptor (Dual-CAR) system and engineered immune cells that target neuroendocrine lineage markers, and methods of use thereof.

### BACKGROUND

Neuroendocrine cancer is a lineage restricted tumor type with predominant expression of neuroendocrine related neuroendocrine and/or neuronal biomarkers. It mainly comprises small cell lung cancer (SCLC), large cell neuroendocrine cancer (LCNC), neuroendocrine prostate cancer (NEPC), pancreatic neuroendocrine tumor (PNET) and gastrointestinal neuroendocrine cancers.

Delta-like ligand 3 (DLL3) is a neuroendocrine lineage marker that is highly expressed in the SCLC and other neuroendocrine tumors but minimally expressed in normal tissues. CD56 is another neuroendocrine lineage marker that has been widely used for the diagnosis of SCLC. In addition to DLL3 and CD56, other neuroendocrine lineage markers include the C-terminal subunit of mucin-1 (MUC1-C), Cadherin-17 (CDH17), disialoganglioside GD2, Netrin-3, Seizure protein 6 homolog (SEZ6), Glypican 2 (GPC2), and B7 Homolog 3 (B7-H3).

SCLC accounts for approximately 15% of all lung cancers. Despite high rates of response to first-line chemotherapy and radiotherapy, patients with extensive-stage disease eventually relapse, and very few patients survive more than 5 years from diagnosis. Treatment options for recurrent or refractory disease are limited, and the treatments that do exist are associated with significant treatment-related toxicities. There is a need to develop treatments for SCLC and other neuroendocrine tumors.

### SUMMARY

Provided herein are compositions and methods that may be used for treating diseases associated with expression of a neuroendocrine lineage marker (e.g., DLL3, CD56, MUCIC, CDH17, GD2, Netrin3, SEZ6, GPC2 or B7-H3). In one aspect, the disclosure relates to engineered immune cells comprising a dual-CAR system targeting neuroendocrine lineage markers, comprising (1) a first CAR comprising a primary intracellular signaling domain of an immune cell and (2) a second CAR that does not comprise a primary intracellular signaling domain derived from CD35 or a primary intracellular signaling domain of an immune cell. In some embodiments, the first CAR specifically recognizes a first antigen and the second CAR specifically recognizes a second antigen. In some embodiments, the first antigen and the second antigen are selected from the group consisting of DLL3, CD56, MUCIC, CDH17, GD2, Netrin3, SEZ6, GPC2 and B7-H3. The disclosure also includes methods of administering the engineered immune cells to the subject.

In one aspect, the disclosure is related to an engineered immune cell comprising a dual chimeric antigen receptor (Dual-CAR) system comprising:
(a) a first chimeric antigen receptor (CAR) comprising:
i) a first antigen binding domain specifically recognizing a first antigen;
ii) a first transmembrane domain; and
iii) a first intracellular signaling domain comprising a co-stimulatory signaling domain of the first intracellular signaling domain; and
(b) a second chimeric antigen receptor (CAR) comprising:
i) a second antigen binding domain specifically recognizing a second antigen;
ii) a second transmembrane domain; and
iii) a second intracellular signaling domain comprising a co-stimulatory signaling domain of the second intracellular signaling domain,
wherein the first intracellular signaling domain further comprises a first primary intracellular signaling domain, and the second intracellular signaling domain does not comprise a primary intracellular signaling domain that is derived from CD3ζ; and
wherein the first antigen and the second antigen are selected from the group consisting of DLL3, CD56, MUCIC, CDH17, GD2, Netrin3, SEZ6, GPC2 and B7-H3.

In one aspect, the disclosure is related to an engineered immune cell comprising a dual chimeric antigen receptor (Dual-CAR) system comprising:
(a) a first chimeric antigen receptor (CAR) comprising:
i) a first antigen binding domain specifically recognizing a first antigen;
ii) a first transmembrane domain; and
iii) a first intracellular signaling domain comprising a co-stimulatory signaling domain of the first intracellular signaling domain; and
(b) a second chimeric antigen receptor (CAR) comprising:
i) a second antigen binding domain specifically recognizing a second antigen;
ii) a second transmembrane domain; and
iii) a second intracellular signaling domain comprising a co-stimulatory signaling domain of the second intracellular signaling domain,
wherein the first intracellular signaling domain further comprises a primary intracellular signaling domain of an immune cell, and the second intracellular signaling domain does not comprise a primary intracellular signaling domain that is derived from CD3ζ; and
wherein each of the first antigen and the second antigen is a neuroendocrine lineage marker.

In some embodiments, the second intracellular signaling domain does not comprise a primary intracellular signaling domain of an immune cell.

In some embodiments, the first primary intracellular signaling domain is derived from CD3ζ.

In some embodiments, the co-stimulatory signaling domain of the first intracellular signaling domain and/or the co-stimulatory signaling domain of the second intracellular signaling domain is derived from a molecule selected from the group consisting of CD27, CD28, 4-1BB, OX40, CD30, CD40, CD3, LFA-1, ICOS, CD2, CD7, LIGHT, NKG2C, B7-H3, and ligands of CD83.

In some embodiments, the co-stimulatory signaling domain of the first intracellular signaling domain comprises a cytoplasmic signaling domain of 4-1BB and the co-stimulatory signaling domain of the second intracellular signaling domain comprises a cytoplasmic signaling domain of CD28.

In some embodiments, the first antigen is same with the second antigen, for example wherein the first antigen in DLL3 and the second antigen is DLL3.

In some embodiments, the first antigen is different from the second antigen.

In some embodiments, the first antigen is selected from the group consisting of DLL3, GD2, and GPC2.

In some embodiments, the second antigen is selected from the group consisting of CD56, MUCIC, CDH17, Netrin3, SEZ6, and B7-H3.

In some embodiments, the first and/or the second binding domain comprise a first antigen binding moiety and/or a second antigen binding moiety selected from a Fab, a Fab′, a F(ab′)2, an Fv, a single-chain Fv (scFv), minibody, a diabody, a single-domain antibody (sdAb) or VHH domain.

In some embodiments, the second antigen is CD56; and/or wherein the second antigen binding domain comprises an anti-CD56 scFv.

In some embodiments, the second antigen is CD56 and the second antigen binding domain comprises an anti-CD56 scFv comprising a VH domain comprising the amino acid sequence of SEQ ID NO: 25; and a VL domain comprising the amino acid sequence of SEQ ID NO: 26.

In some embodiments, the anti-CD56 scFv comprises:
a VH domain comprising a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3, wherein the HCDRs are defined according to the Kabat numbering scheme; and
a VL domain comprising a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, wherein the LCDRs are defined according to the Kabat numbering scheme;

In some embodiments, the anti-CD56 scFv comprises:
a VH domain comprising a HCDR1 comprising the amino acid sequence of SEQ ID NO: 13, a HCDR2 comprising the amino acid sequence of SEQ ID NO: 14, and a HCDR3 comprising the amino acid sequence of SEQ ID NO: 15, wherein the HCDRs are defined according to the AbM numbering scheme; and
a VL domain comprising a LCDR1 comprising the amino acid sequence of SEQ ID NO: 16, a LCDR2 comprising the amino acid sequence of SEQ ID NO: 17, and a LCDR3 comprising the amino acid sequence of SEQ ID NO: 18, wherein the LCDRs are defined according to the AbM numbering scheme.

In some embodiments, the first antigen is DLL3; and/or wherein the first antigen binding domain comprises one or more anti-DLL3 sdAbs.

In some embodiments, the first antigen binding domain comprises two anti-DLL3 sdAbs, the two anti-DLL3 sdAbs comprising:
a first anti-DLL3 sdAb comprising a CDR1 comprising the amino acid sequence of SEQ ID NO: 7, a CDR2 comprising the amino acid sequence of SEQ ID NO: 8, and a CDR3 comprising the amino acid sequence of SEQ ID NO: 9, wherein the CDRs are defined according to the Kabat numbering scheme; and
a second anti-DLL3 sdAb comprising a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12, wherein the CDRs are defined according to the Kabat numbering scheme.

In some embodiments, the first antigen binding domain comprises two anti-DLL3 sdAbs, the two anti-DLL3 sdAbs comprising:
a first anti-DLL3 sdAb comprising a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21, wherein the CDRs are defined according to the AbM numbering scheme; and
a second anti-DLL3 sdAb comprising a CDR1 comprising the amino acid sequence of SEQ ID NO: 22, a CDR2 comprising the amino acid sequence of SEQ ID NO: 23, and a CDR3 comprising the amino acid sequence of SEQ ID NO: 24, wherein the CDRs are defined according to the AbM numbering scheme.

In some embodiments, the first antigen binding domain comprises a first anti-DLL3 sdAb comprising the amino acid sequence of SEQ ID NO: 28, and a second anti-DLL3 sdAb comprising the amino acid sequence of SEQ ID NO: 29.

In some embodiments, the first and/or the second transmembrane domain comprises a transmembrane domain that is derived from a molecule selected from the group consisting of CD8a, CD4, CD28, 4-1BB, CD80, CD86, CD152 and PD1.

In some embodiments, the first transmembrane domain and/or the second transmembrane domain comprise an amino acid sequence that is at least 95%, 99%, or 100% identical to the amino acid sequence set forth in any one of SEQ ID NOs: 34-36.

In some embodiments, the first CAR comprises a hinge domain located between the C-terminus of the first antigen binding domain and the N-terminus of the first transmembrane domain; and wherein the hinge domain of the first chimeric antigen receptor is derived from CD8a or CD28; and the second CAR comprises a hinge domain located between the C-terminus of the second antigen binding domain and the N-terminus of the second transmembrane domain; and wherein the hinge domain of the second chimeric antigen receptor is derived from CD8a or CD28.

In some embodiments, the hinge domain of the first CAR and/or the hinge domain of the second CAR comprises an amino acid sequence that is at least 95%, 99%, or 100% identical to the amino acid sequence set forth in any one of SEQ ID NOs: 31-33.

In some embodiments, the hinge domain of the second CAR comprises the amino acid sequence of SEQ ID NO: 33 or SEQ ID NO:32, and/or wherein the second transmembrane domain comprises the amino acid sequence of SEQ ID NO: 36 or SEQ ID NO: 35.

In some embodiments, each of the first CAR comprises a signal peptide located at the N-terminus of the first antigen binding domain, optionally wherein the signal peptide is derived from CD8a; and/or the second CAR comprises a signal peptide located at the N-terminus of the second antigen binding domain, optionally wherein the signal peptide is derived from CD8a.

In some embodiments, the first CAR comprises an amino acid sequence that is at least 95%, 99%, or 100% identical to the amino acid sequence of SEQ ID NO: 40; and/or wherein the second CAR comprises an amino acid sequence that is at least 95%, 99%, or 100% identical to the amino acid sequence of SEQ ID NO: 41 or SEQ ID NO: 42.

In some embodiments, the engineered immune cell comprises a polypeptide comprising the first CAR and/or the second CAR having an amino acid sequence selected from a group consisting of SEQ ID NOs: 40-44.

In one aspect, the disclosure is related to a nucleic acid comprising one or more nucleic acid sequences that encode a first chimeric antigen receptor (CAR) and a second chimeric antigen receptor (CAR), wherein
(a) the first chimeric antigen receptor (CAR) comprising:
i) an antigen binding domain specifically recognizing a first antigen;
ii) a first transmembrane domain; and
iii) a first intracellular signaling domain comprising a co-stimulatory signaling domain of the first intracellular signaling domain; and
(b) the second chimeric antigen receptor (CAR) comprising:
i) a second antigen binding domain specifically recognizing a second antigen;
ii) a second transmembrane domain; and
iii) a second intracellular signaling domain comprising a co-stimulatory signaling domain of the second intracellular signaling domain,
wherein the first intracellular signaling domain further comprises a first primary intracellular signaling domain of an immune cell, and the second intracellular signaling domain does not comprise a primary intracellular signaling domain that is derived from CD3ζ; and
wherein the first antigen and the second antigen are selected from the group consisting of DLL3, CD56, MUCIC, CDH17, GD2, Netrin3, SEZ6, GPC2 and B7-H3.

In some embodiments, the second intracellular signaling domain does not comprise a primary intracellular signaling domain of an immune cell.

In some embodiments, the nucleic acid comprises one or more nucleic acid sequences encoding:
a. the first CAR having an amino acid sequence at least 95%, 99%, or 100% identical to the amino acid of SEQ ID NO: 40; and/or
b. the second CAR having an amino acid sequence at least 95%, 99%, or 100% identical to the amino acid of SEQ ID NO: 41, or SEQ ID NO: 42; or
c. the first CAR and the second CAR having an amino acid sequence at least 95%, 99%, or 100% identical to the amino acid of SEQ ID NO: 43 or SEQ ID NO: 44.

In some embodiments, the nucleic acid comprises a first nucleic acid sequence encoding the first CAR and a second nucleic acid sequence encoding the second CAR, wherein
(i) the first nucleic acid and the second nucleic acid are operably linked to the same promoter;
(ii) the first nucleic acid is upstream of the second nucleic acid or wherein the first nucleic acid is downstream of the second nucleic acid; and/or
(iii) the first nucleic acid and the second nucleic acid are connected via a nucleic acid sequence encoding a linker sequence of P2A or T2A.

In one aspect, the disclosure is related to a single chimeric antigen receptor (CAR) comprising:
(a) an antigen binding domain comprising an anti-CD56 binding moiety specifically recognizing CD56;
(b) a transmembrane domain; and
(c) an intracellular signaling domain,
wherein the anti-CD56 binding moiety comprises a VH domain comprising the amino acid sequence of SEQ ID NO: 25; and a VL domain comprising the amino acid sequence of SEQ ID NO: 26.

In some embodiments, the anti-CD56 binding moiety comprises:
a VH domain comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3, wherein the HCDRs are defined according to the Kabat numbering scheme; and
a VL domain comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, wherein the LCDRs are defined according to the Kabat numbering scheme;

In some embodiments, the anti-CD56 binding moiety comprises:
a VH domain comprises a HCDR1 comprising the amino acid sequence of SEQ ID NO: 13, a HCDR2 comprising the amino acid sequence of SEQ ID NO: 14, and a HCDR3 comprising the amino acid sequence of SEQ ID NO: 15, wherein the HCDRs are defined according to the AbM numbering scheme; and
a VL domain comprises a LCDR1 comprising the amino acid sequence of SEQ ID NO: 16, a LCDR2 comprising the amino acid sequence of SEQ ID NO: 17, and a LCDR3 comprising the amino acid sequence of SEQ ID NO: 18, wherein the LCDRs are defined according to the AbM numbering scheme.

In some embodiments, the intracellular signaling domain comprises a co-stimulatory signaling domain and does not comprise a primary intracellular signaling domain of an immune cell.

In some embodiments, the co-stimulatory signaling domain comprises an amino acid sequence of a cytoplasmic domain that is at least 95% identical to an amino acid sequence of a cytoplasmic domain of CD28 and/or 4-1BB.

In some embodiments, the transmembrane domain is derived from a molecule selected from the group consisting of CD8a, CD4, CD28, CD137, CD80, CD86, CD152 and PD1.

In some embodiments, the single CAR further comprises a hinge domain derived from CD8a or CD28.

In some embodiments, the single CAR further comprises a signal peptide domain derived from CD8a.

In some embodiments, the hinge domain is at least 95% identical to the amino acid sequence set forth in any one of SEQ ID NOs: 31-33, and/or the transmembrane domain is at least 95% identical to the amino acid sequence set forth in any one of SEQ ID NOs: 34-36, and/or the signal peptide domain is at least 95% identical to the amino acid sequence of SEQ ID NO: 30.

In some embodiments, the single CAR comprises an amino acid sequence that is at least 95%, 99%, or 100% identical to the amino acid sequence set forth in SEQ ID NO: 41 or SEQ ID NO: 42.

In one aspect, the disclosure is related to a nucleic acid comprising one or more nucleic acid sequences that encode the single CAR described herein.

In one aspect, the disclosure is related to a vector comprising the nucleic acid described herein.

In one aspect, the disclosure is related to the use of the vector described herein to produce an engineered immune cell.

In one aspect, the disclosure is related to an engineered immune cell comprising the single CAR described herein, or the nucleic acid described herein, or the vector described herein.

In some embodiments, the engineered immune cell is selected from the group consisting of T cell, αβT cell, γδT cell, NK cell, peripheral blood mononuclear cell (PBMC), hematopoietic stem cell, pluripotent stem cell, an embryonic stem cell, and a combination thereof.

In one aspect, the disclosure is related to a pharmaceutical composition comprising the engineered immune cell described herein, and a pharmaceutically acceptable carrier.

In one aspect, the disclosure is related to a method for producing the engineered immune cell described herein, the method comprising introducing the vector described herein into a cell.

In one aspect, the disclosure is related to a method of treating a subject having cancer or at risk of having cancer, the method comprising: administering the engineered immune cell described herein, the pharmaceutical composition described herein, or the engineered immune cell described herein into a subject having cancer or at risk of having cancer.

In some embodiments, the subject has a neuroendocrine tumor (NET).

In some embodiments, the cancer is small cell lung cancer (SCLC), large cell neuroendocrine cancer (LCNC), neuroendocrine prostate cancer (NEPC), pancreatic neuroendocrine tumor (PNET) or gastrointestinal neuroendocrine cancers.

In one aspect, the disclosure is related to a dual CAR system comprising:
(a) a first CAR comprising:
i) a first antigen binding domain specifically recognizing a first antigen;
ii) a first transmembrane domain; and
iii) a first intracellular signaling domain comprising a co-stimulatory signaling domain of the first intracellular signaling domain; and
(b) a second CAR comprising:
i) a second antigen binding domain specifically recognizing a second antigen;
ii) a second transmembrane domain; and
iii) a second intracellular signaling domain comprising a co-stimulatory signaling domain of the second intracellular signaling domain,
wherein the first intracellular signaling domain further comprises a first primary intracellular signaling domain, and the second intracellular signaling domain does not comprise a primary intracellular signaling domain that is derived from CD3ζ; and
wherein the first antigen and the second antigen are selected from the group consisting of DLL3, CD56, MUCIC, CDH17, GD2, Netrin3, SEZ6, GPC2 and B7-H3.

### DESCRIPTION OF DRAWINGS

FIGS. 1A-1C show schematic illustrations of DLL3 bi-specific chimeric antigen receptor (DLL3 CAR) and DLL3/CD56 dual chimeric antigen receptors (DLL3/CD56 Dual-CARs). FIG. 1A shows an exemplary structure of a DLL3 CAR. FIG. 1B shows an exemplary structure of a DLL3/CD56 Dual-CAR. FIG. 1C shows another exemplary structure of a DLL3/CD56 Dual-CAR. DLL3/CD56 Dual-CAR-1 #(FIG. 1B) and DLL3/CD56 Dual-CAR-2 #(FIG. 1C) are coupled with divergent downstream stimulatory signals.

FIGS. 2A-2D show the expression of CAR comprising anti-DLL3 VHHs by human primary T cells. FIG. 2A shows CAR expression by T cells transfected with a DLL3 CAR. FIG. 2B shows CAR expression by T cells transfected with DLL3 CAR DLL3/CD56 Dual-CAR-1 #. FIG. 2C shows CAR expression by T cells transfected with a DLL3 CAR DLL3/CD56 Dual-CAR-2 #. FIG. 2C shows CAR expression by un-transfected control T cells. The X-axis shows anti-V_{H}H signals (anti-V_{H}H) and the Y-axis shows cell size signals (FSC-H).

FIGS. 3A-3C show the cytotoxicity analysis of CAR-T cells against tumor cells at different E:T ratios. DLL3 CAR or DLL3/CD56 Dual-CARs were co-cultured with wild type SHP-77 (FIG. 3A), SHP-77-Luc-DLL3 KO (FIG. 3B) or NK-92 cells (FIG. 3C) at the E:T ratio of 0.5:1 and/or 2:1, respectively. The CAR-T mediated target cell lysis was analyzed by examining the LDH release in the co-culture supernatant. SHP-77 cells are DLL3 and CD56 double positive. SHP-77-Luc-DLL3 KO cells are DLL3 negative and CD56 positive. NK-92 cells are DLL3 positive and CD56 negative.

FIGS. 4A-4D show the IFN-γ release by CAR-T cells. IFN-γ released by DLL3 CAR-cells and DLL3/CD56 Dual-CAR-T cells co-cultured with wild type SHP-77 (FIG. 4A), SHP-77-Luc-DLL3 KO (FIG. 4B) or NK-92 cells (FIG. 4C) were analyzed, respectively. Baseline IFN-γ release by CAR-T and UnT cells were measured in T cell culture alone without any target cells (FIG. 4D). A stands for below the lower limit of quantification (<LLOQ).

FIGS. 5A-5C show the CAR-T persistence (FIG. 5A) and CAR-T expansion (FIGS. 5B-5C) of DLL3 CAR and DLL3/CD56 Dual-CAR-T cells. CAR-T cells and UnT cells were cultured with tumor cells at an E:T ratio of 1:5 in a repetitive tumor challenge assay.

FIGS. 6A-6D show the in vivo efficacy and pharmacokinetics of DLL3 CAR-T cells and DLL3/CD56 Dual-CAR-T cells in an xenograft model based on SCLC tumor cell line NCI-H82. FIG. 6A shows tumor volume changes as an indicator for efficacy of CAR-T cells. FIG. 6B shows body weight changes as an indicator for the safety of CAR-T cells. Expansion of total T and CAR-T cells in peripheral blood of NCG mice were observed 14 days post treatment as an indicator for the pharmacokinetics of the CAR-T cells (FIGS. 6C-6D). FIG. 6C shows total T cell expansion. FIG. 6D shows CAR-T expansion.

FIGS. 7A-7C show the CAR-T persistence (FIG. 7A) and CAR-T expansion (FIGS. 7B-7C) of DLL3 biCAR and DLL3/CD56 Dual-biCAR-T cells. CAR-T cells and UnT cells were cultured with tumor cells at an E:T ratio of 1:5 in a repetitive tumor challenge assay.

FIG. 8 shows the sequences listed in the present application.

### DETAILED DESCRIPTION

Provided herein are compositions and methods for treating diseases associated with expression of a neuroendocrine lineage marker (e.g., DLL3, CD56, MUCIC, CDH17, GD2, Netrin3, SEZ6, GPC2 or B7-H3). In one aspect, the disclosure relates to a dual chimeric antigen receptor (Dual-CAR) system targeting neuroendocrine lineage markers, the system comprises (1) a first CAR comprising a primary intracellular signaling domain of an immune cell and (2) a second CAR that does not comprise a primary intracellular signaling domain derived from CD35 or a primary intracellular signaling domain of an immune cell. The first CAR specifically recognizes a first antigen and the second CAR specifically recognizes a second antigen. The first antigen and the second antigen may be selected from the group consisting of DLL3, CD56, MUCIC, CDH17, GD2, Netrin3, SEZ6, GPC2 and B7-H3. In one aspect, the disclosure relates to engineered immune cells comprising the dual-CAR system.

In the present disclosure, a dual-CAR targeting strategy is employed to deliver multiple inputs to drive the expansion and cytotoxicity of immune cells (e.g., T cells). The dual-CAR targeting strategy delivered by DLL3 and CD56 in the present disclosure demonstrate the superiority of dual-CAR-T cells targeting two antigens (e.g., DLL3 and CD56) in comparison to CAR-T cells targeting a single antigen (e.g., DLL3). In addition, due to the predominant expression of CD56 in the neuronal lineages, classical approach of coupling of CD56 binding to co-stimulatory signaling domain together with CD3ζ (CD3 zeta) signaling domain can be optimized by removing the CD3ζ signaling domain.

As used herein, the term “derived from” when made in reference to a domain described herein refers to a domain that is obtained from the relevant functional portion of a protein (e.g., by recombinant expression or de novo synthesis). The term encompasses domains with naturally occurring sequences and sequences with mutations. A domain derived from a particular protein can have a sequence that is at least 80%, 85%, 90%, 95%, 98%, 99%, or 100% identical to the relevant functional portion of the particular protein. A domain derived from a particular protein can be from a natural or a synthetic source.

As used herein, a “vector” is any construct capable of delivering one or more nucleic acids of interest to a host cell when the vector is introduced to the host cell. An “expression vector” is capable of delivering and expressing the one or more nucleic acids of interest as an encoded polypeptide in a host cell into which the expression vector has been introduced. Thus, in an expression vector, the nucleic acid of interest is positioned for expression in the vector by being operably linked with regulatory elements such as a promoter, enhancer, and/or a poly-A tail, either within the vector or in the genome of the host cell at or near or flanking the integration site of the nucleic acid of interest such that the nucleic acid of interest will be translated in the host cell introduced with the expression vector.

As used herein, the term “chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” A CAR may comprise an extracellular ligand binding domain or an extracellular antigen binding domain specific for one or more ligands or antigens (such as tumor antigens), a transmembrane domain, and an intracellular signaling domain. “CAR-T cell” refers to a T cell that expresses a CAR.

As used herein, the term “antibody” includes, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. Antibodies also include, but are not limited to, synthetic antibodies, recombinantly produced antibodies, single domain antibodies including from Camelidae species (e.g., llama or alpaca) or their humanized variants, intrabodies, anti-idiotypic (anti-Id) antibodies, and functional fragments (e.g., antigen-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments) include single-chain Fv (scFv) (e.g., including monospecific, bispecific, etc.), VHH fragment, Fab fragment, F(ab′) fragment, F(ab)2 fragment, F(ab′)2 fragment, disulfide-linked Fvs (dsFv), Fd fragment, Fv fragment, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). An antibody may contain an Fc region of a human antibody.

“Single-chain Fv” also abbreviated as “sFv” or “scFv” are antibody fragments that comprise the VH and VL domains connected into a single polypeptide chain. For example, the scFv polypeptide further comprises a polypeptide linker between the VH and VL domains which enables the scFv to form the desired structure for antigen binding. For a review of the scFv, see Pluckthun in The Pharmacology of Monoclonal Antibodies, vol. 113, Rosenburg and Moore eds., Springer-Verlag, New York, pp. 269-315 (1994).

“Single domain antibody” or “sdAb” as used herein refers to a single monomeric variable antibody domain and which is capable of antigen binding (e.g., single domain antibodies that bind to DLL3 or CD56). Single domain antibodies include VHH domains as described herein. Examples of single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. For example, a single domain antibody can be derived from antibodies raised in Camelidae species, for example in camel, llama, dromedary, alpaca and guanaco, as described herein. Other species besides Camelidae may produce heavy chain antibodies naturally devoid of light chain; VHHs derived from such other species are within the scope of the disclosure. The single domain antibody (e.g., VHH or VHH) provided herein has a structure of FR1-CDR1-FR2-CDR2-FR3-CDR3-FR4. Single domain antibodies may be genetically fused or chemically conjugated to another molecule (e.g., an agent) as described herein. Single domain antibodies may be part of a bigger binding molecule (e.g., a multispecific antibody or a chimeric antigen receptor).

As used herein, the terms “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences. CDR regions are well known to those skilled in the art and have been defined by well-known numbering systems/schemes. For example, the Kabat Complementarity Determining Regions (CDRs) are based on sequence variability and are the most commonly used (see, e.g., Kabat et al., supra). Chothia refers instead to the location of the structural loops (see. e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27 (1): 55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309:657-70 (2001). Correspondence between the numbering system/scheme, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in Table 1 below.

TABLE 1
Exemplary CDRs According to Various Numbering Systems

| Loop | Kabat | AbM | Chothia | Contact | IMGT |
|---|---|---|---|---|---|
| CDR-L1 | L24--L34 | L24--L34 | L26--L32 or | L30--L36 | L27--L38 |
| | | | L24--L34 | | |
| CDR-L2 | L50--L56 | L50--L56 | L50--L52 or | L46--L55 | L56--L65 |
| | | | L50--L56 | | |
| CDR-L3 | L89--L97 | L89--L97 | L91--L96 or | L89--L96 | L105-L117 |
| | | | L89--L97 | | |
| CDR-H1 | H31--H35B | H26--H35B | H26-- | H30--H35B | H27--H38 |
| | (Kabat | | H32 . . . 34 | | |
| | Numbering) | | | | |
| CDR-H1 | H31--H35 | H26--H35 | H26--H32 | H30--H35 | |
| | (Chothia | | | | |
| | Numbering) | | | | |
| CDR-H2 | H50--H65 | H50--H58 | H53--H55 or | H47--H58 | H56--H65 |
| | | | H52--H56 | | |
| CDR-H3 | H95--H102 | H95--H102 | H96--H101 or | H93--H101 | H105-H117 |
| | | | H95--H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, AbM, Chothia, or Contact method. In some instances, one or more positions according to the Kabat numbering may not be occupied in the actual sequence, or the actual sequence may contain more amino acid residues than the number allowed for by the Kabat numbering. See, e.g., Deschacht et al., 2010. J Immunol 184:5696-704 for an exemplary numbering for VHH domains according to Kabat. In other cases, the particular amino acid sequence of a CDR is given. It should be noted CDR regions may also be defined by a combination of various numbering systems, e.g., a combination of Kabat and Chothia numbering systems, a combination of Kabat and AbM numbering systems, or a combination of Kabat and IMGT numbering systems. Therefore, the term such as “a CDR as set forth in a specific VH or VHH” includes any CDRs as defined by the exemplary CDR numbering systems described above, but is not limited thereby. Once a variable region (e.g., a VHH domain, a VH or VL domain) is given, those skilled in the art would understand that CDRs within the region can be defined by different numbering systems or combinations thereof.

As used herein, the term “antigen binding domain” or “extracellular antigen binding domain” refers to a portion of a full-length antibody, wherein the portion of the antibody is capable of specifically binding to an antigen. An antigen binding fragment may comprise at least one variable domain (e.g., a variable domain of a heavy chain, single domain antibody or VHH). Non-limiting examples of antibody fragments include, e.g., Fab, Fab′, F(ab′)2, and Fv fragments.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, MEGALIGN™ (DNASTAR), Snapgene or Clustal W software, or such other similar software program. Those skilled in the art can determine appropriate parameters for measuring alignment, including any algorithms needed to achieve maximal alignment over the full length of the sequences being compared.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, endocrine systems and neuroendocrine tumor (NET); as well as adenocarcinomas which include malignancies such as most colon cancers, renal-cell carcinoma, prostate cancer and/or testicular tumors, small cell lung cancer (SCLC), large cell neuroendocrine cancer (LCNC), neuroendocrine prostate cancer (NEPC), pancreatic neuroendocrine tumor (PNET), gastrointestinal neuroendocrine cancers, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

As used herein, the term “subject” and “patient” are used interchangeably throughout the specification and describe an animal, human or non-human, to whom treatment according to the methods of the present disclosure is provided. Veterinary and non-veterinary applications are contemplated by the present disclosure. Human subjects can be adult humans or juvenile humans (e.g., humans below the age of 18 years old). In addition to humans, subjects include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### 1. Neuroendocrine Cancer and Neuroendocrine Lineage Markers

The neuroendocrine system has various cells distributed in non-endocrine functional structures, able to synthesize amines and peptides with both local (paracrine) and systemic (endocrine) effects. The presence of neuroendocrine lineage markers is commonly associated to the occurrence of neuroendocrine cancers.

Delta-like ligand 3 (DLL3) is a neuroendocrine lineage marker. It is an inhibitory Notch ligand that is highly expressed in neuroendocrine tumors and also in small cell lung cancer (SCLC), but minimally expressed in normal tissues. DLL3 is an inhibitory Notch pathway ligand that is highly upregulated and aberrantly expressed on the cell surface in SCLC and other high-grade neuroendocrine tumors. Notch signaling is downregulated during neuroendocrine tumor growth and is inhibited by DLL3 expression. DLL3 expression is regulated by achaete-scute homolog 1 (ASCL1), a transcription factor that is required for proper development of pulmonary neuroendocrine cells and is an oncogenic driver in SCLC. In preclinical models, DLL3 expression promotes SCLC migration and invasion through a mechanism that involves control of the epithelial-mesenchymal transition protein Snail. DLL3 is specifically expressed on the surface of SCLC tumor cells. DLL3 is also expressed in other tumor types of neuroendocrine origin, including melanoma, glioblastoma multiforme, small cell bladder cancer, metastatic castration-resistant prostate cancer, and neuroendocrine lung tumors.

CD56, also known as neural cell adhesion molecule (NCAM), is a member of the immunoglobulin superfamily engaged in both so-called homophilic and heterophilic interactions. It is a neuroendocrine lineage marker. Three main isoforms exist of CD56 (NCAM-120, NCAM-140, and NCAM-180), all generated by alternative splicing from one single gene, differing in their intracellular domain length. CD56 is often considered a marker of neural lineage commitment due to its discovery site. However, CD56 expression is also found in, among others, the hematopoietic system. In addition, the expression of CD56 is most stringently associated with, but certainly not limited to, natural killer (NK) cells. CD56 has been detected on other lymphoid cells, including gamma delta (γδ) T cells and activated CD8+ T cells, as well as on dendritic cells (DCs). Also, in the bone marrow, at the site where hematopoiesis occurs, CD56 fulfills a pivotal role. Mesenchymal stromal cells provide niches for hematopoietic stem cells by, inter alia, the expression of adhesion molecules comprising CD56, maintaining long-term hematopoiesis.

Other neuroendocrine lineage markers include the C-terminal subunit of mucin-1 (MUC1-C), Cadherin-17 (CDH17), disialoganglioside GD2, Netrin-3, Seizure protein 6 homolog (SEZ6), Glypican 2 (GPC2), and B7 Homolog 3 (B7-H3).

More information regarding neuroendocrine cancer and neuroendocrine lineage markers can be found in, e.g., Ferolla, P., et al. “The biological characterization of neuroendocrine tumors: the role of neuroendocrine markers.” Journal of endocrinological investigation 31 (2008): 277-286; Rekhtman, Natasha. “Lung neuroendocrine neoplasms: recent progress and persistent challenges.” Modern Pathology 35. Suppl 1 (2022): 36-50; Van Acker, Heleen H., et al. “CD56 in the immune system: more than a marker for cytotoxicity?” Frontiers in immunology 8 (2017): 892; Owen, Dwight H., et al. “DLL3: an emerging target in small cell lung cancer.” Journal of hematology & oncology 12.1 (2019): 1-8; Yao, James, et al. “DLL3 as an Emerging Target for the Treatment of Neuroendocrine Neoplasms.” The Oncologist 27.11 (2022): 940-951; each of which is incorporated by reference in its entirety.

### 2. Engineered Immune Cells Comprising Chimeric Antigen Receptors

One aspect of the present application provides engineered immune cells comprising chimeric antigen receptors (CARs) targeting neuroendocrine lineage markers, wherein the chimeric antigen receptors may be dual chimeric antigen receptors (Dual-CARs) comprising (1) a first CAR comprising a primary intracellular signaling domain of an immune cell and (2) a second CAR that does not comprise a primary intracellular signaling domain derived from CD3ζ or a primary intracellular signaling domain of an immune cell. Another aspect of the present application provides an engineered immune cell comprising a single CAR targeting CD56.

### 2.1 Chimeric Antigen Receptors

### 2.1.1 Dual-CAR

One aspect of the present application provides a dual chimeric antigen receptor (Dual-CAR) system comprising: (a) a first chimeric antigen receptor (CAR) comprising:
i) a first antigen binding domain specifically recognizing a first antigen;
ii) a first transmembrane domain; and
iii) a first intracellular signaling domain comprising a co-stimulatory signaling domain of the first intracellular signaling domain; and
(b) a second chimeric antigen receptor (CAR) comprising:
i) a second antigen binding domain specifically recognizing a second antigen;
ii) a second transmembrane domain; and
iii) a second intracellular signaling domain comprising a co-stimulatory signaling domain of the second intracellular signaling domain; wherein the first intracellular signaling domain further comprises a primary intracellular signaling domain of an immune cell, and the second intracellular signaling domain does not comprise a primary intracellular signaling domain that is derived from CD34. In some embodiments, each of the first antigen and the second antigen is a neuroendocrine lineage marker. In some embodiments, the first antigen and the second antigen are selected from the group consisting of DLL3, CD56, MUCIC, CDH17, GD2, Netrin3, SEZ6, GPC2 and B7-H3.

In some embodiments, the first antigen and the second antigen are same, for example the first antigen and the second antigen are DLL3. In some embodiments, the first antigen and the second antigen are different. The dual-CAR system may comprise a first CAR that specifically binds to a neuroendocrine lineage marker selected from the group consisting of DLL3, GD2, and GPC2, for example DLL3. The dual-CAR system may comprise a second CAR that specifically binds to a neuroendocrine lineage marker selected from the group consisting of CD56, MUCIC, CDH17, Netrin3, SEZ6, and B7-H3, for example CD56. The dual-CAR system may comprise a first CAR which specifically binds to DLL3 and a second CAR which specifically binds to CD56.

### Antigen Binding Domains of Dual-CARs

The antigen binding domain of each CAR in the dual-CAR system described herein may comprise one or more (such as any one of 1, 2, 3, 4, 5, 6 or more) antibodies or antibody fragments.

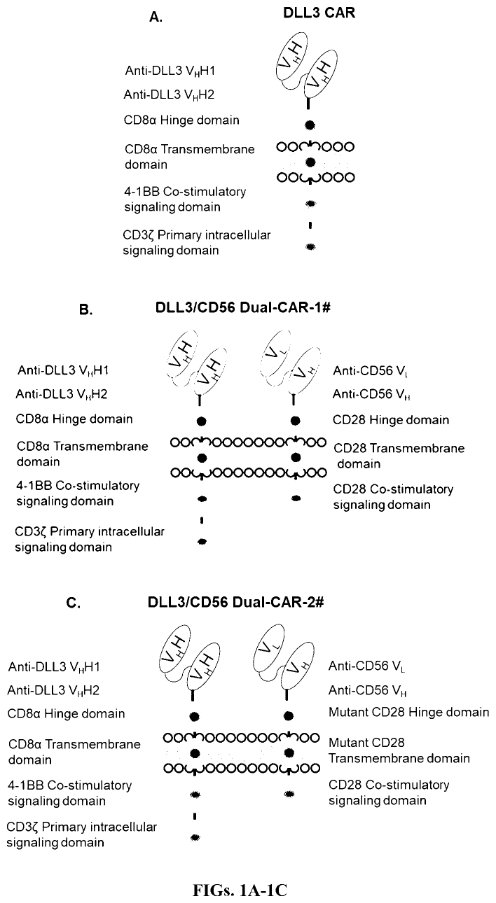

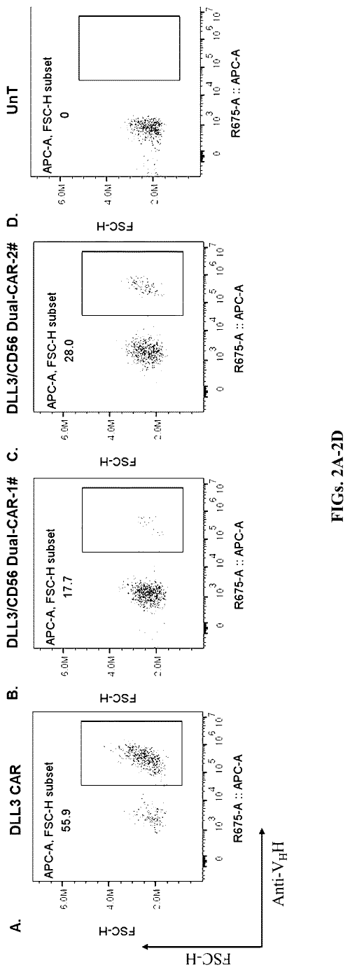

The first and/or the second antigen binding domain may comprise a first antigen binding moiety and/or a second antigen binding moiety, which can be selected from a Fab, a Fab′, a F(ab′)2, an Fv, a single-chain Fv (scFv), minibody, a diabody, a single-domain antibody (sdAb) or VHH domain. The first antigen binding domain may comprise a first and a second antigen binding moieties which are sdAbs (e.g., VHH domains). The second antigen binding domain may comprise an antigen binding moiety which is a single chain Fv (scFv, such as VL-VH pairs). In some embodiments, the first CAR comprises a first antigen binding domain comprising two anti-DLL3 sdAbs (e.g., VHH domains). In some embodiments, the second CAR comprises a second antigen binding domain comprising an anti-CD56 scFv (e.g., VL-VH pairs). The antigen binding domain of the first CAR and/or the second CAR described herein may comprise one or more (such as any one of 1, 2, 3, 4, 5, 6 or more) sdAbs or scFvs. The sdAbs and/or scFvs can be fused to each other directly via peptide bonds, or via peptide linkers. Exemplary structures of dual-CARs are shown in FIGS. 1A-1C.

In some embodiments, the present disclosure provides, in the dual CAR system, a first CAR targeting DLL3 (also referred herein as “DLL3 CAR”) comprising: (a) a first antigen binding domain comprising an anti-DLL3 binding moiety; (b) a first transmembrane domain; and (c) a first intracellular signaling domain, wherein the first intracellular signaling domain comprises a co-stimulatory signaling domain and a primary intracellular signaling domain of an immune effector cell (such as T cell). In some embodiments, the primary intracellular signaling domain is derived from CD3. The co-stimulatory signaling domain of the first intracellular signaling domain may be derived from a molecule selected from the group consisting of a co-stimulatory molecule derived from CD27, CD28, CD137 (4-1BB), OX40, CD30, CD40, CD3, LFA-1, ICOS, CD2, CD7, LIGHT, NKG2C, B7-H3, and ligands of CD83. In some embodiments, the DLL3 CAR further comprises a hinge domain (such as a CD8a hinge domain) located between the C-terminus of the DLL3 antigen binding domain and the N-terminus of the transmembrane domain. In some embodiments, the DLL3 CAR further comprises a signal peptide (such as a CD8a signal peptide) located at the N-terminus of the antigen binding domain. In some embodiments, the first CAR includes the polypeptide comprising from the N-terminus to the C-terminus: a CD8a signal peptide, a DLL3 antigen binding domain, a CD8a hinge domain, a CD8a transmembrane domain, a co-stimulatory signaling domain derived from 4-1BB, and a primary intracellular signaling domain derived from CD35. The DLL3 CAR may be monospecific. The DLL3 CAR may be bispecific or bivalent. A wide variety of antigen binding domain sequences can be used as the antigen binding domain of the DLL3 CAR, such as AMG119 and AMG157, and those disclosed in different patents e.g., WO2019200007A1, WO2020180591A1, and WO2021008610A1.

In some embodiments, the present disclosure provides, in the dual-CAR system, a first CAR targeting DLL3 (also referred herein as “DLL3 CAR”) comprising: (a) a first antigen binding domain comprising an anti-DLL3 binding moiety comprising two anti-DLL3 sdAbs (e.g., VHH domains); (b) a first transmembrane domain; and (c) a first intracellular signaling domain, wherein the two anti-DLL3 sdAbs comprise any one of the following: (1) a first anti-DLL3 sdAb comprising a CDR1 comprising the amino acid sequence of SEQ ID NO: 7, a CDR2 comprising the amino acid sequence of SEQ ID NO: 8, and a CDR3 comprising the amino acid sequence of SEQ ID NO: 9; and a second anti-DLL3 sdAb comprising a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12, wherein the CDRs are defined according to the Kabat numbering scheme; or (2) a first anti-DLL3 sdAb comprising a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21; and a second anti-DLL3 sdAb comprising a CDR1 comprising the amino acid sequence of SEQ ID NO: 22, a CDR2 comprising the amino acid sequence of SEQ ID NO: 23, and a CDR3 comprising the amino acid sequence of SEQ ID NO: 24, wherein the CDRs are defined according to the AbM numbering scheme. The anti-DLL3 sdAb may be camelid, chimeric, human, or humanized. The VHH CDRs (CDR1-3) may be determined according to the Kabat numbering scheme, the IMGT numbering scheme, the AbM numbering scheme, the Chothia numbering scheme, the Contact numbering scheme, or a combination thereof. In some embodiments, the anti-DLL3 sdAb comprises a VHH domain comprising an amino acid sequence selected from the group consisting of SEQ ID NOs: 28-29. In some embodiments, the anti-DLL3 binding moiety comprises a first VHH domain comprising the amino acid sequence of SEQ ID NO: 28, or at least 80%, 85%, 90%, or 95% identical to the amino acid sequence set forth in SEQ ID NO: 28; and a second VHH domain comprising the amino acid sequence of SEQ ID NO: 29, or at least 80%, 85%, 90%, or 95% identical to the amino acid sequence set forth in SEQ ID NO: 29. In some embodiments, the first intracellular signaling domain comprises a co-stimulatory signaling domain and a primary intracellular signaling domain of an immune effector cell (such as T cell). In some embodiments, the primary intracellular signaling domain is derived from CD35. The co-stimulatory signaling domain of the first intracellular signaling domain may be derived from a molecule selected from the group consisting of a co-stimulatory molecule derived from CD27, CD28, CD137 (4-1BB), OX40, CD30, CD40, CD3, LFA-1, ICOS, CD2, CD7, LIGHT, NKG2C, B7-H3, and ligands of CD83. In some embodiments, the DLL3 CAR further comprises a hinge domain (such as a CD8a hinge domain) located between the C-terminus of the DLL3 antigen binding domain and the N-terminus of the transmembrane domain. In some embodiments, the DLL3 CAR further comprises a signal peptide (such as a CD8a signal peptide) located at the N-terminus of the antigen binding domain. In some embodiments, the first CAR includes the polypeptide comprising from the N-terminus to the C-terminus: a CD8a signal peptide, the DLL3 antigen binding domain, a CD8a hinge domain, a CD8a transmembrane domain, a co-stimulatory signaling domain derived from 4-1BB, and a primary intracellular signaling domain derived from CD35.

In some embodiments, the first CAR (e.g., DLL3 CAR) comprises an amino acid sequence that is at least 95%, 99%, or 100% identical to the amino acid sequence of SEQ ID NO: 40. In some embodiments, the first CAR (e.g., DLL3 CAR) comprises the amino acid sequence of SEQ ID NO: 40. Also provided is a polypeptide comprising the amino acid sequence of SEQ ID NO: 40.

In some embodiments, the present disclosure provides, in the dual-CAR system, a second CAR targeting CD56 (also referred herein as “CD56 CAR”) comprising: (a) a second antigen binding domain comprising an anti-CD56 binding moiety; (b) a second transmembrane domain; and (c) a second intracellular signaling domain comprising a co-stimulatory signaling domain, wherein the second intracellular signaling domain does not comprise a primary intracellular signaling domain that is derived from CD32. In some embodiments, the second intracellular signaling domain does not comprise a primary intracellular signaling domain of an immune effector cell (such as T cell). The anti-CD56 binding moiety can be derived from one or more variable regions of anti-CD56 antibody, such as clone HCD56 (BioLegend), clone MEM-188 (BioLegend), the humanized anti-CD56 antibody lorvotuzumab (huN901), or those disclosed in patent application such as WO2017023859A1.

The co-stimulatory signaling domain of the second intracellular signaling domain may be derived from a molecule selected from the group consisting of a co-stimulatory molecule derived from CD27, CD28, CD137 (4-1BB), OX40, CD30, CD40, CD3, LFA-1, ICOS, CD2, CD7, LIGHT, NKG2C, B7-H3, and ligands of CD83. In some embodiments, the CD56 CAR further comprises a hinge domain (such as a CD28 hinge domain or a mutant CD28 hinge domain) located between the C-terminus of the CD56 antigen binding domain and the N-terminus of the transmembrane domain. In some embodiments, the CD56 CAR further comprises a signal peptide (such as a CD8a signal peptide) located at the N-terminus of the CD56 antigen binding domain. In some embodiments, the second CAR includes the polypeptide comprising from the N-terminus to the C-terminus: a CD8a signal peptide, the CD56 antigen binding domain, a CD28 hinge domain or a mutant CD28 hinge domain, a transmembrane domain derived from CD28, and a co-stimulatory signaling domain derived from CD28.

In some embodiments, the present disclosure provides a second CAR targeting CD56 (also referred herein as “CD56 CAR”) in the dual-CAR system comprising: (a) a second antigen binding domain comprising an anti-CD56 binding moiety comprising an anti-CD56 scFv (e.g., VL-VH pairs); (b) a second transmembrane domain; and (c) a second intracellular signaling domain comprising a co-stimulatory signaling domain, wherein the second intracellular signaling domain does not comprise a primary intracellular signaling domain that is derived from CD34, and wherein the anti-CD56 scFv comprises any one of the following: (a) a VH domain comprising a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; and a VL domain comprising a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, wherein the CDRs are defined according to the Kabat numbering scheme; or (b) a VH domain comprising a HCDR1 comprising the amino acid sequence of SEQ ID NO: 13, a HCDR2 comprising the amino acid sequence of SEQ ID NO: 14, and a HCDR3 comprising the amino acid sequence of SEQ ID NO: 15; and a VL domain comprising a LCDR1 comprising the amino acid sequence of SEQ ID NO: 16, a LCDR2 comprising the amino acid sequence of SEQ ID NO: 17, and a LCDR3 comprising the amino acid sequence of SEQ ID NO: 18, wherein the CDRs are defined according to the AbM numbering scheme. The anti-CD56 scFv may be mouse, chimeric, human, or humanized. The VH CDRs (HCDR1-3) and VL CDRs (LCDR1-3) may be determined according to the Kabat numbering scheme, the IMGT numbering scheme, the AbM numbering scheme, the Chothia numbering scheme, the Contact numbering scheme, or a combination thereof. In some embodiments, the anti-CD56 scFv comprises a VH domain comprising the amino acid sequence of SEQ ID NO: 25, or at least 80%, 85%, 90%, or 95% identical to the amino acid sequence set forth in SEQ ID NO: 25, and a VL domain comprising the amino acid sequence of SEQ ID NO: 26, or at least 80%, 85%, 90%, or 95% identical to the amino acid sequence set forth in SEQ ID NO: 26. In some embodiments, the second intracellular signaling domain does not comprise a primary intracellular signaling domain of an immune effector cell (such as T cell). The co-stimulatory signaling domain of the second intracellular signaling domain may be derived from a molecule selected from the group consisting of a co-stimulatory molecule derived from CD27, CD28, CD137 (4-1BB), OX40, CD30, CD40, CD3, LFA-1, ICOS, CD2, CD7, LIGHT, NKG2C, B7-H3, and ligands of CD83. In some embodiments, the CD56 CAR further comprises a hinge domain (such as a CD28 hinge domain or a mutant CD28 hinge domain) located between the C-terminus of the CD56 antigen binding domain and the N-terminus of the second transmembrane domain. In some embodiments, the CD56 CAR further comprises a signal peptide (such as a CD8a signal peptide) located at the N-terminus of the CD56 antigen binding domain. In some embodiments, the first CAR includes the polypeptide comprising from the N-terminus to the C-terminus: a CD8a signal peptide, the CD56 antigen binding domain, a CD28 hinge domain or a mutant CD28 hinge domain, a transmembrane domain derived from CD28, and a co-stimulatory signaling domain derived from CD28.

In some embodiments, the second CAR (e.g., CD56 CAR) in the dual-CAR system comprises an amino acid sequence that is at least 95%, 99%, or 100% identical to an amino acid sequence set forth in any one of SEQ ID NOs: 41-42. In some embodiments, the second CAR (e.g., CD56 CAR) in the dual-CAR system comprises an amino acid sequence set forth in any one of SEQ ID NOs: 41-42. Also provided is a polypeptide comprising an amino acid sequence selected from a group consisting of SEQ ID NOs: 41-42.

### 2.1.2 Single CAR

In one aspect, the present disclosure provides a single chimeric antigen receptor (CAR) comprising: (a) an extracellular antigen binding domain that specifically recognizes an antigen; (b) a transmembrane domain; and (c) an intracellular signaling domain, wherein the antigen is a neuroendocrine lineage marker. In some embodiments, the antigen is selected from DLL3, CD56, MUCIC, CDH17, GD2, Netrin3, SEZ6, GPC2 or B7-H3. In some embodiments, the antigen is CD56. In some embodiments, the antigen is DLL3.

### Antigen Binding Domain of Single CAR

The antigen binding domain may be derived from one or more variable regions of a monoclonal antibody linked together, such as a single-chain variable fragment (scFv) or VHH domain. An antibody in scFv format consists of variable regions of heavy chain (VH) and light chain (VL), which are joined together by a flexible peptide linker or a disulfide bond. The antigen binding domain may comprise one or more (e.g., 1, 2, 3, 4, 5, or 6) VH-VL pairs. The antigen binding domain may comprise one or more (e.g., 1, 2, 3, 4, 5, or 6) VL-VH pairs.

In some embodiments, the present disclosure provides a single CAR targeting CD56 (also referred herein as “CD56 CAR”) comprising: (a) an antigen binding domain comprising an anti-CD56 binding moiety specifically recognizing CD56; (b) a transmembrane domain; and (c) an intracellular signaling domain, wherein the anti-CD56 binding moiety comprises a VH domain comprising the amino acid sequence of SEQ ID NO: 25, or at least 80%, 85%, 90%, or 95% identical to the amino acid sequence of SEQ ID NO: 25; and a VL domain comprising the amino acid sequence of SEQ ID NO: 26, or at least 80%, 85%, 90%, or 95% identical to the amino acid sequence of SEQ ID NO: 26. The CD56 antigen binding domain thereof may contain an anti-CD56 scFv sequence that is at least 80%, 85%, 90%, or 95% identical to a selected scFv sequence set forth in SEQ ID NO: 27. The anti-CD56 scFv may be mouse, chimeric, human, or humanized.

The CDRs of anti-CD56 binding moiety or anti-CD56 scFv, e.g., VH CDRs (HCDR1-3) and VL CDRs (LCDR1-3) may be determined according to the Kabat numbering scheme, the IMGT numbering scheme, the AbM numbering scheme, the Chothia numbering scheme, the Contact numbering scheme, or a combination thereof. The VH CDRs of anti-CD56 binding moiety or anti-CD56 scFv may comprise the amino acid sequences of SEQ ID NOs: 1-3, and VL CDRs of anti-CD56 binding moiety or anti-CD56 scFv may comprise the amino acid sequences of SEQ ID NOs: 4-6 as defined by Kabat numbering scheme. The VH CDRs of anti-CD56 binding moiety or anti-CD56 scFv may comprise the amino acid sequences of SEQ ID NOs: 13-15, and VL CDRs of anti-CD56 binding moiety or anti-CD56 scFv may comprise the amino acid sequences of SEQ ID NOs: 16-18. The anti-CD56 binding moiety or anti-CD56 scFv may comprise (a) a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; and (b) a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, wherein the CDRs are defined according to the Kabat numbering scheme. The anti-CD56 binding moiety or anti-CD56 scFv may comprise (a) a HCDR1 comprising the amino acid sequence of SEQ ID NO: 13, a HCDR2 comprising the amino acid sequence of SEQ ID NO: 14, and a HCDR3 comprising the amino acid sequence of SEQ ID NO: 15; and (b) a LCDR1 comprising the amino acid sequence of SEQ ID NO: 16, a LCDR2 comprising the amino acid sequence of SEQ ID NO: 17, and a LCDR3 comprising the amino acid sequence of SEQ ID NO: 18, wherein the CDRs are defined according to the AbM numbering scheme. The anti-CD56 binding moiety thereof described herein may contain a VH containing the HCDR1 with zero, one or two amino acid insertions, deletions, and/or substitutions; the HCDR2 with zero, one or two amino acid insertions, deletions, and/or substitutions; and/or the HCDR3 with zero, one or two amino acid insertions, deletions, and/or substitutions. The anti-CD56 binding moiety thereof described herein may contain a VL containing the LCDR1 with zero, one or two amino acid insertions, deletions, and/or substitutions; the LCDR2 with zero, one or two amino acid insertions, deletions, and/or substitutions; and/or the LCDR3 with zero, one or two amino acid insertions, deletions, and/or substitutions.

In some embodiments, the CD56 CAR may comprise: (a) an antigen binding domain comprising an anti-CD56 scFv (e.g., VL-VH pairs); (b) a transmembrane domain; and (c) an intracellular signaling domain, wherein the anti-CD56 scFv comprises any one of the following: (a) a VH domain comprising a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; and a VL domain comprising a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, wherein the CDRs are defined according to the Kabat numbering scheme; or (b) a VH domain comprising a HCDR1 comprising the amino acid sequence of SEQ ID NO: 13, a HCDR2 comprising the amino acid sequence of SEQ ID NO: 14, and a HCDR3 comprising the amino acid sequence of SEQ ID NO: 15; and a VL domain comprising a LCDR1 comprising the amino acid sequence of SEQ ID NO: 16, a LCDR2 comprising the amino acid sequence of SEQ ID NO: 17, and a LCDR3 comprising the amino acid sequence of SEQ ID NO: 18, wherein the CDRs are defined according to the AbM numbering scheme. In some embodiments, the anti-CD56 scFv comprises a VH domain comprising the amino acid sequence of SEQ ID NO: 25, or at least 80%, 85%, 90%, or 95% identical to the amino acid sequence set forth in SEQ ID NO: 25, and a VL domain comprising the amino acid sequence of SEQ ID NO: 26, or at least 80%, 85%, 90%, or 95% identical to the amino acid sequence set forth in SEQ ID NO: 26. The intracellular signaling domain may comprise a co-stimulatory signaling domain and a primary intracellular signaling domain of an immune effector cell (such as T cell). In some embodiments, the intracellular signaling domain may not comprise a primary intracellular signaling domain of an immune effector cell (such as T cell). The co-stimulatory signaling domain of the intracellular signaling domain may be derived from a molecule selected from the group consisting of CD27, CD28, CD137 (4-1BB), OX40, CD30, CD40, CD3, LFA-1, ICOS, CD2, CD7, LIGHT, NKG2C, B7-H3, and ligands of CD83. In some embodiments, the CD56 CAR further comprises a hinge domain (such as a CD28 hinge domain or mutant CD28 hinge domain) located between the C-terminus of the CD56 antigen binding domain and the N-terminus of the transmembrane domain. In some embodiments, the CD56 CAR further comprises a signal peptide (such as a CD8a signal peptide) located at the N-terminus of the CD56 antigen binding domain. In some embodiments, the CD56 CAR includes the polypeptide comprising from the N-terminus to the C-terminus: a CD8a signal peptide, the CD56 antigen binding domain, a CD28 hinge domain or a mutant CD28 hinge domain, a transmembrane domain derived from CD28, and a co-stimulatory signaling domain derived from CD28.

The CD56 CAR may comprise an amino acid sequence that is at least 95%, 99%, or 100% identical to an amino acid sequence set forth in any one of SEQ ID NOs: 41-42. The CD56 CAR may comprise an amino acid sequence set forth in any one of SEQ ID NOs: 41-42. Also provided is a polypeptide comprising an amino acid sequence set forth in any one of SEQ ID NOs: 41-42.

### 2.2 Other CAR Regions

Other components and regions of dual-CAR and single CAR are described in more detail below.

### Intracellular Signaling Domain

The dual-CAR (including the first and the second CARs) and single CAR of the present disclosure comprise at least one intracellular signaling domain. The intracellular signaling domain is responsible for activation of at least one of the normal effector functions of the immune cell expressing the CARs. As used herein, the terms “intracellular signaling domain”, or “intracellular signaling region” are used interchangeably herein and refer to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal (e.g., at least 50%, 60%, 70%, 80%, 85%, 90%, 95%, 96%, 97%, 98%, or 99% of the entire portion). An intracellular signaling domain can be derived from the intracellular or cytoplasmic signaling domain of a wild-type membrane protein (e.g., a receptor) or a functional variant thereof. An intracellular signaling domain may have one or more mutations, including e.g., insertions, deletions, and/or substitutions. An intracellular signaling domain may comprise a co-stimulatory signaling domain and/or a primary intracellular signaling domain of an immune effector cell. The term “effector function” refers to a specialized function of a cell. Effector function of a T cell, for example, may be cytolytic activity or helper activity including the secretion of cytokines.

In some embodiments, the intracellular signaling domain may comprise a primary intracellular signaling domain of an immune effector cell. The CAR may comprise an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. The primary intracellular signaling domain may contain a signaling motif known as immunoreceptor tyrosine-based activation motif, or ITAM. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix (6-8) YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary intracellular signaling sequences include those derived from CD3ζ, FcR gamma (FCER1G), FcR beta (Fc Epsilon Rib), CD3 gamma, CD3 delta, CD3 epsilon, CD5, CD22, CD79a, CD79b, and/or CD66d.

The primary intracellular signaling domain of the single CAR, and/or the primary intracellular signaling domain of the first CAR in the dual-CAR system may be derived from CD3ζ, and retain the relevant function of the primary intracellular signaling domain of CD35. The primary intracellular signaling domain may have a sequence that is identical to the primary intracellular signaling domain of CD3ζ, or at least 80%, 85%, 90%, 95%, 98% or 99% identical to the sequence of the primary intracellular signaling domain of CD3ζ. The primary intracellular signaling domain may consist of the cytoplasmic signaling domain of CD3ζ. The primary intracellular signaling domain may be a cytoplasmic signaling domain of wild-type CD3ζ. The primary intracellular signaling domain may be a functional mutant of the cytoplasmic signaling domain of CD3ζ containing one or more mutations, such as Q65K. The primary intracellular signaling domain of CD3ζ may comprise an amino acid sequence that is at least 80%, at least 85%, at least 90%, at least 95%, or 100% identical to the amino acid sequence of SEQ ID NO: 39 or 54.

### Co-Stimulatory Signaling Domain

Many immune effector cells require co-stimulation, in addition to stimulation of an antigen-specific signal, to promote cell proliferation, differentiation and survival, as well as to activate effector functions of the cell. The CAR may comprise at least one co-stimulatory signaling domain. The term “co-stimulatory signaling domain,” as used herein, refers to at least a portion of a protein that mediates signal transduction within a cell to induce an immune response such as an effector function. The co-stimulatory signaling domain of the chimeric antigen receptor described herein can be a cytoplasmic signaling domain from a co-stimulatory protein, which transduces a signal and modulates responses mediated by immune cells, such as T cells, NK cells, macrophages, neutrophils, or eosinophils. The co-stimulatory signaling domain can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. An intracellular signaling domain may comprise two or more (such as about any of 2, 3, 4, or more) co-stimulatory signaling domains. An intracellular signaling domain may comprise two or more of the same co-stimulatory signaling domains. An intracellular signaling domain may comprise two or more co-stimulatory signaling domains from different co-stimulatory proteins, such as any two or more co-stimulatory proteins described herein. In some embodiments, the intracellular signaling domain comprises a primary intracellular signaling domain (such as primary intracellular signaling domain of CD3ζ) and one or more co-stimulatory signaling domains. In some embodiments, the one or more co-stimulatory signaling domains and the primary intracellular signaling domain (such as primary intracellular signaling domain of CD3ζ) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as primary intracellular signaling domain of CD3ζ). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CARs described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect). Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thyl, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCLIA, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C.

In some embodiments, the one or more co-stimulatory signaling domains are derived from one or more molecules selected from the group consisting of CD27, CD28, CD137 (4-1BB), OX40, CD30, CD40, CD3, lymphocyte function-associated antigen-1 (LFA-1), CD2, CD7, LIGHT, NKG2C, B7-H3 and ligands that specially bind to CD83.

In some embodiments, the intracellular signaling domain of the CAR (e.g., dual-CAR or single CAR) of the present disclosure comprises a co-stimulatory signaling domain derived from CD137 (i.e., 4-1BB). In some embodiments, the intracellular signaling domain of the CAR (e.g., the first CAR in the dual-CAR or single CAR) comprises a primary intracellular signaling domain of CD35 and a co-stimulatory signaling domain of 4-1BB. In some embodiments, the intracellular signaling domain of the CAR (e.g., dual-CAR or single CAR) comprises a co-stimulatory signaling domain of 4-1BB. The 4-1BB co-stimulatory signaling domain may comprise an amino acid sequence that is at least 80%, at least 85%, at least 90%, at least 95%, or 100% identical to the amino acid sequence of SEQ ID NO: 37.

In some embodiments, the intracellular signaling domain of the CAR (e.g., dual-CAR or single CAR) of the present disclosure comprises a co-stimulatory signaling domain derived from CD28. The intracellular signaling domain of the CAR (e.g., the first CAR in the dual-CAR or single CAR) may comprise a primary intracellular signaling domain of CD3 and a co-stimulatory signaling domain of CD28. In some embodiments, the intracellular signaling domain of the CAR (e.g., dual-CAR or single CAR) comprises a co-stimulatory signaling domain of CD28. The CD28 co-stimulatory signaling domain may comprise an amino acid sequence that is at least 80%, at least 85%, at least 90%, at least 95%, or 100% identical to the amino acid sequence of SEQ ID NO: 38.

Also within the scope of the present disclosure are variants of any of the co-stimulatory signaling domains described herein, such that the co-stimulatory signaling domain is capable of modulating the immune response of the immune cell. The co-stimulatory signaling domains may comprise up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

In the dual-CAR system, the first intracellular signaling domain of the first CAR may consist of 1, 2, 3, 4, 5, or more co-stimulatory signaling domains. The first intracellular signaling domain of the first CAR may consist of one co-stimulatory signaling domain. The first intracellular signaling domain of the first CAR may comprise 1, 2, 3, 4, 5, or more primary intracellular signaling domain of an immune cell (such as T cell). The first intracellular signaling domain of the first CAR may consist of one primary intracellular signaling domain of an immune cell (such as T cell), which is capable of inducing a primary activation signal in an immune cell (such as T cell). A primary intracellular signaling domain may be a T cell receptor (TCR) component. A primary intracellular signaling domain of the first intracellular signaling domain may comprise an immunoreceptor tyrosine-based activation motif (ITAM). A primary intracellular signaling domain of the first intracellular signaling domain may comprise an amino acid sequence derived from CD3 zeta, FcR gamma, FcR beta, CD3 gamma, CD3 delta, CD3 epsilon, CD5, CD22, CD79a, CD79b, CD278 (ICOS), FceRI, CD66d, DAP10, DAP12, or combinations thereof. In some embodiments, the primary intracellular signaling domain of the first intracellular signaling domain is derived from CD3ζ. The primary intracellular signaling domain may consist of the cytoplasmic signaling domain of CD3ζ. The primary intracellular signaling domain may be a cytoplasmic signaling domain of wild-type CD3ζ. The primary intracellular signaling domain may be a functional mutant of the cytoplasmic signaling domain of CD3ζ containing one or more mutations, such as Q65K. The primary intracellular signaling domain of CD3ζ may comprise an amino acid sequence that is at least 80%, at least 85%, at least 90%, at least 95%, or 100% identical to the amino acid sequence of SEQ ID NO: 39 or 54.

In the dual-CAR system, the second intracellular signaling domain of the second CAR may consist of 1, 2, 3, 4, 5, or more co-stimulatory signaling domains. The second intracellular signaling domain of the second CAR may consist of one co-stimulatory signaling domain. The second intracellular signaling domain of the second CAR may each comprise more than one co-stimulatory domain.

Each of the first CAR and the second CAR in the dual-CAR system as described herein may comprise at least one co-stimulatory signaling domain of the first intracellular signaling domain and/or one co-stimulatory signaling domain of the second intracellular signaling domain, which may comprise a functional signaling domain from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1, CD11a/CD18, 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD 11b, ITGAX, CD 11c, ITGB1, CD29, ITGB2, CD18, LFA-1, ITGB7, NKG2D, NKG2C, TNFR2, TRANCE/RANKL, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a CD83 ligand. The co-stimulatory signaling domain of the first intracellular signaling domain and/or the co-stimulatory signaling domain of the second intracellular signaling domain may comprise a functional signaling domain from OX40, CD28, 4-1BB, ICOS, or a signaling portion thereof. The co-stimulatory signaling domain of the first intracellular signaling domain and/or the second intracellular signaling domain may derived from 4-1BB and/or CD28. In some embodiments, the co-stimulatory signaling domain of the first intracellular signaling domain comprises a cytoplasmic signaling domain of 4-1BB and the co-stimulatory signaling domain of the second intracellular signaling domain comprises a cytoplasmic signaling domain of CD28. The 4-1BB co-stimulatory signaling domain may comprise an amino acid sequence that is at least 80%, at least 85%, at least 90%, at least 95%, or 100% identical to the amino acid sequence of SEQ ID NO: 37. The CD28 co-stimulatory signaling domain may comprise an amino acid sequence that is at least 80%, at least 85%, at least 90%, at least 95%, or 100% identical to the amino acid sequence of SEQ ID NO: 38.

### Transmembrane Domain

Each of the first CAR and the second CAR in the dual-CAR system and the single CAR of the present disclosure comprise a transmembrane domain that can be directly or indirectly fused to the extracellular antigen binding domain. As used herein, the terms “transmembrane domain” or “transmembrane region” are used interchangeably herein to refer to the portion of a membrane protein (e.g., a receptor) that is embedded in the cell membrane. A transmembrane region can be entire portion of the protein that is embedded in the cell membrane, or just a part thereof (e.g., at least 50%, 60%, 70%, 80%, 85%, 90%, 95%, 96%, 97%, 98%, or 99% of the entire portion). A transmembrane region can be derived from the transmembrane region of a wild-type receptor or a functional variant thereof. A transmembrane region can have one or more mutations, including e.g., insertions, deletions, and/or substitutions. The transmembrane domain may be derived either from a natural or from a synthetic source. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane.

Transmembrane domains are classified based on three dimensional structures of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N-terminus and C-terminus.

A transmembrane domain of each CAR described herein may be derived from a Type I single-pass membrane protein. The transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. The N-terminus and the C-terminus of a multi-pass membrane protein may be present on opposing sides of the lipid bilayer, e.g., the N-terminus of the protein is present on the cytoplasmic side of the lipid bilayer and the C-terminus of the protein is present on the extracellular side.

The transmembrane domain of each CAR may comprise a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD152, CD154, KIRDS2, OX40, CD2, CD27, LFA-1 (CDI 1a, CD18), ICOS (CD278), 4-1BB (CD137), GITR, CD40, BAFFR, HVEM (LIGHTR), SLAMF7, NKp80 (KLRFI), CD160, CD19, IL-2R beta, IL-2R gamma, IL-7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAMI (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Lyl08), SLAM (SLAMF1, CD150, IPO-3), BLAME (SLAMF8), SELPLG (CD162), LTBR, PAG/Cbp, NKp44, NKp30, NKp46, NKG2D, PD1, and/or NKG2C. In some embodiments, the transmembrane domain is derived from a molecule selected from the group consisting of CD8α, CD4, CD28, 4-1BB, CD80, CD86, CD152 and PD1.

In some embodiments, the transmembrane domain is derived from CD8α. The transmembrane domain may be a CD8α transmembrane domain comprising the amino acid sequence of SEQ ID NO: 34, or a sequence that is at least 80%, 85%, 90%, or 95% identical to the amino acid sequence of SEQ ID NO: 34. In some embodiments, the transmembrane region domain is derived from CD28. The transmembrane domain may be a CD28 transmembrane domain comprising the amino acid sequence of SEQ ID NO: 35, or a sequence that is at least 80%, 85%, 90%, or 95% identical to the amino acid sequence of SEQ ID NO: 35. The transmembrane domain may be a mutant CD28 transmembrane domain comprising the amino acid sequence of SEQ ID NO: 36, or a sequence that is at least 80%, 85%, 90%, or 95% identical to the amino acid sequence of SEQ ID NO: 36.

Transmembrane domains for use in the CARs described herein can also comprise at least a portion of a synthetic, non-naturally occurring protein segment. The transmembrane domain may be a synthetic, non-naturally occurring alpha helix or beta sheet. The protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, helps to orient the transmembrane domain in the lipid bilayer. One or more cysteine residues may be present in the transmembrane region of the transmembrane domain. One or more cysteine residues are present in the cytoplasmic region of the transmembrane domain. The cytoplasmic region of the transmembrane domain may comprise positively charged amino acids. In some embodiments, the cytoplasmic region of the transmembrane domain comprises the amino acids arginine, serine, and lysine.

The transmembrane region of the transmembrane domain may comprise hydrophobic amino acid residues. A transmembrane domain of each CAR provided herein may comprise an artificial hydrophobic sequence. For example, a triplet of phenylalanine, tryptophan and valine may be present at the C terminus of the transmembrane domain. A transmembrane region comprises mostly hydrophobic amino acid residues, such as alanine, leucine, isoleucine, methionine, phenylalanine, tryptophan, or valine. The transmembrane region may comprise a poly-leucine-alanine sequence. The hydropathy, or hydrophobic or hydrophilic characteristics of a protein or protein segment, can be assessed by any method known in the art, for example the Kyte and Doolittle hydropathy analysis.

### Hinge Domain

Each of the first CAR and the second CAR in the dual-CAR system and the single CAR of the present may comprise a hinge domain that is located between the extracellular antigen binding domain and the transmembrane domain. As used herein, the terms “hinge domain” or “hinge region” are used interchangeably herein to refer to the portion of a membrane protein (e.g., a receptor) that connects a transmembrane region and the extracellular domain. A hinge domain is a small structural domain that sits between the antigen binding domain and the cell's outer membrane. For example, a hinge domain may be located between a C-terminus of an antigen binding domain and an N-terminus of a transmembrane domain. A hinge domain can be part of an extracellular domain. A hinge domain can be derived from a hinge domain of a wild-type receptor or a functional variant thereof. A hinge domain can have one or more mutations, including e.g., insertions, deletions, and/or substitutions.

A hinge domain can allow for flexibility of the protein and movement of one or both of the domains relative to one another. Any amino acid sequence that provides such flexibility and movement of the extracellular antigen binding domain relative to the transmembrane domain of the effector molecule can be used.

The hinge domain may contain about 10-100 amino acids, e.g., about any one of 15-75 amino acids, 20-50 amino acids, or 30-60 amino acids. The hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

The hinge domain can be a hinge domain of a naturally occurring protein. Hinge domains of any protein known in the art to comprise a hinge domain can be used in the chimeric receptors described herein. The hinge domain may be a portion of a hinge domain of a naturally occurring protein and confers flexibility to the chimeric receptor. A hinge domain may be derived from CD8α. The hinge domain may be a portion of the hinge domain of CD8α, e.g., a fragment containing at least 15 (e.g., 20, 25, 30, 35, or 40) consecutive amino acids of the hinge domain of CD8α. The CD8a hinge domain may comprise the amino acid sequence of SEQ ID NO: 31, or may comprise an amino acid sequence that is at least 80%, at least 85%, at least 90%, at least 95%, or 100% identical to the amino acid sequence of SEQ ID NO: 31. A hinge domain may be a membrane-proximal region of CD28. The hinge domain of CD28 may be a wild-type CD28 hinge domain or a mutant CD28 hinge domain. The CD28 hinge domain may comprise the amino acid sequence of SEQ ID NO: 32, or may comprise an amino acid sequence that is at least 80%, at least 85%, at least 90%, at least 95%, or 100% identical to the amino acid sequence of SEQ ID NO: 32. The mutant CD28 hinge domain may comprise the amino acid sequence of SEQ ID NO: 33, or may comprise an amino acid sequence that is at least 80%, at least 85%, at least 90%, at least 95%, or 100% identical to the amino acid sequence of SEQ ID NO: 33.

Hinge domains of antibodies, such as an IgG, IgA, IgM, IgE, or IgD antibodies, can also be compatible for use in the pH-dependent chimeric receptor systems described herein. The hinge domain may be a hinge domain that joins the constant domains CH1 and CH2 of an antibody. A hinge domain may be of an antibody and may comprise the hinge domain of the antibody and one or more constant regions of the antibody. The hinge domain may comprise a hinge domain of an antibody and the CH3 constant region of the antibody. The hinge domain may comprise a hinge domain of an antibody and the CH2 and CH3 constant regions of the antibody. The antibody may be an IgG, IgA, IgM, IgE, or IgD antibody. The antibody may be an IgG1, IgG2, IgG3, or IgG4 antibody. The hinge region may comprise the hinge region and the CH2 and CH3 constant regions of an IgG1 antibody. The hinge region may comprise the hinge region and the CH3 constant region of an IgG1 antibody.

Non-naturally occurring peptides may also be used as hinge domains for the chimeric receptors described herein. The hinge domain between the C-terminus of the extracellular ligand-binding domain of an Fc receptor and the N-terminus of the transmembrane domain is a peptide linker, such as a (GxS)n linker, wherein x and n, independently can be an integer between 3 and 12, including 3, 4, 5, 6, 7, 8, 9, 10, 11, 12, or more.

### Signal Peptide

Each of the first CAR and the second CAR in the dual-CAR system and the single CAR of the present disclosure may comprise a signal peptide (also known as a signal sequence) at the N-terminus of the antigen binding domain. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. A signal peptide may be derived from a molecule selected from the group consisting of CD8α, GM-CSF receptor a, and IgG1 heavy chain. The signal peptide may be a signal peptide derived from CD8α. The signal peptide may comprise an amino acid sequence that is at least 80%, at least 85%, at least 90%, at least 95%, or 100% identical to the amino acid sequence of SEQ ID NO: 30.

### Peptide Linker

When multiple antibodies (e.g., multiple antibody fragments) are present in the present CARs, the various antibodies or antibody fragments may be fused to each other via one or more peptide linkers (e.g., VHH-Linker-VHH, or VL-Linker-VH). The antibodies or antibody fragments are directly fused to each other without any peptide linkers. The peptide linkers connecting different antibodies may be the same or different. Different domains of the CARs may also be fused to each other via peptide linkers.

Each peptide linker in a CAR may have the same or different length and/or sequence depending on the structural and/or functional features of the antibodies and/or the various domains. Each peptide linker may be selected and optimized independently. The length, the degree of flexibility and/or other properties of the peptide linker(s) used in the CARs may have some influence on properties, including but not limited to the affinity, specificity or avidity for one or more particular antigens or epitopes. For example, longer peptide linkers may be selected to ensure that two adjacent domains do not sterically interfere with one another. A short peptide linker may be disposed between the transmembrane domain and the intracellular signaling domain of a CAR. A peptide linker may comprise flexible residues (such as glycine and serine) so that the adjacent domains are free to move relative to each other. For example, a glycine-serine doublet can be a suitable peptide linker.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long.

The peptide linker may have a naturally occurring sequence, or a non-naturally occurring sequence. For example, a sequence derived from the hinge region of heavy chain only antibodies may be used as the linker. See, for example, WO1996/34103. The peptide linker may be a flexible linker. Exemplary flexible linkers include but not limited to glycine polymers (G) n, glycine-serine polymers, glycine-alanine polymers, alanine-serine polymers, threonine-serine, and other flexible linkers known in the art. Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the CARs provided herein, the disclosure of each of which is incorporated herein by reference.

Various linker sequences are known in the art, including, without limitation, glycine serine (GS) spacers (also known as GS linkers) such as (GS)n, (SG)n, (GSGGS)n and (GGGS)n, where n represents an integer of at least 1. Exemplary linker sequences can comprise amino acid sequences including, without limitation, GGGGSGGGGSGGGGS (SEQ ID NO: 47), GGSG (SEQ ID NO: 48), GGSGG (SEQ ID NO: 49), GSGSG (SEQ ID NO: 50), GSGGG (SEQ ID NO: 51), GGGSG (SEQ ID NO: 52), GSSSG (SEQ ID NO: 53), and the like. Those of skill in the art would be able to select the appropriate linker sequence.

The dual-CAR system may include a first CAR and a second CAR linked by a self-cleaving peptide. As used herein, a “self-cleaving peptide” or “2A linker” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A linkers are known to those of skill in the art, including, without limitation, those found in members of the Picornaviridae virus family, e.g., foot-and-mouth disease virus (FMDV), equine rhinitis A virus (ERAVO, Thosea asigna virus (TaV), and porcine tescho virus-1 (PTV-1); and carioviruses such as Theilovirus and encephalomyocarditis viruses. 2A linkers derived from FMDV, ERAV, PTV-1, and TaV are referred to herein as “F2A,” “E2A,” “P2A,” and “T2A,” respectively. Those of skill in the art would be able to select the appropriate self-cleaving peptide. In some embodiments, the self-cleaving peptide is a P2A linker that has an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 45. In some embodiments, the self-cleaving peptide is a T2A linker that has an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 46.

The dual-CAR system may comprise a DLL3 CAR and a CD56 CAR, linked by a self-cleaving peptide. The dual-CAR system may comprise a DLL3 CAR comprising an amino acid sequence that is at least 95%, 99%, or 100% identical to the amino acid sequence of SEQ ID NO: 40, and a CD56 CAR comprising an amino acid sequence that is at least 95%, 99%, or 100% identical to the amino acid sequence of SEQ ID NOs: 41-42. In some embodiments, the dual-CAR system may comprise a polypeptide encoding the DLL3 CAR and the CD56 CAR with a 2A linker comprising an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 43 or SEQ ID NO: 44.

The hinge domain, transmembrane domain, and/or intracellular signaling domain (e.g., costimulatory signaling domain and/or primary intracellular signaling domain) of the CARs may be derived from a first generation, a second generation, a third generation, or a fourth generation CAR structure. Details of the structural features of CARs can be found, e.g., in Jackson, Hollie J., et al., Nature Reviews Clinical Oncology 13.6 (2016): 370; and Subklewe, Marion, et al., Transfusion Medicine and Hemotherapy 46.1 (2019): 15-24; each of which is incorporated herein by reference.

### 2.3 Engineered Immune Cells

In one aspect, the present disclosure provides engineered immune cells (e.g., T cells, NK cells, tumor-infiltrating lymphocytes) that comprise the dual-CAR described herein. The engineered immune cells may comprise the single CAR (e.g., CD56 CAR) described herein. In some embodiments, the engineered immune cells can be used to treat various disorder or disease as described herein (e.g., a neuroendocrine cancer).

The cell that is to be engineered can be obtained from e.g., humans or non-human animals. The cell that is to be engineered can be obtained from bacteria, fungi, humans, rats, mice, rabbits, monkeys, pig or any other species. For example, the cell can be obtained from humans, rats or mice. The cells may be mouse lymphocytes and engineered (e.g., transduced) to express the CAR described herein. The cell may be obtained from humans. The cell may be a blood cell. The cell may be a leukocyte (e.g., a T cell), lymphocyte or any other suitable blood cell type. The cell may be a peripheral blood cell. The cell may be a tumor-infiltrating lymphocyte (TIL). The cell may be a T cell, a B cell or an NK cell. The cells may be human peripheral blood mononuclear cells (PBMCs). The human PBMCs may be CD3+ cells. The human PBMCs may be CD8+ cells or CD4+ cells.

The cell may be a T cell. The T cells may express one or more CARs that recognizes a specific antigen on the surface of a target cell. T cells can be obtained by various methods known in the art, e.g., in vitro culture of T cells (e.g., tumor infiltrating lymphocytes) isolated from subjects. Genetically engineered T cells can be obtained by transducing T cells (e.g., isolated from the peripheral blood of subjects), with a vector, such as the vector provided herein. The T cells may be CD4+ T cells, CD8+ T cells, or regulatory T cells. The T cells may be T helper type 1 T cells and/or T helper type 2 T cells. The T cell expressing the CAR may be an αβ-T cell. The T cell expressing this receptor may be a γδ-T cell. The T cells may be central memory T cells. The T cells may be effector memory T cells. The T cells may be naïve T cells.

The preparation of the engineered immune cells may include one or more culture and/or preparation steps. The cells which are to be engineered to express the one or more CARs, can be isolated from a sample, such as a biological sample, e.g., one obtained from or derived from a subject. The subject from which the cell is isolated may have a disease or condition or need a cell therapy or to which cell therapy will be administered. The subject may be a human in need of a particular therapeutic intervention, such as an adoptive cell therapy for which the cells are being isolated, processed, and/or engineered.

The cells may be stem cells, such as multipotent and pluripotent stem cells, including induced pluripotent stem cells (iPSCs). The cells can be primary cells, such as those isolated directly from a subject and/or isolated from a subject and frozen. The stem cells may be cultured with additional differentiation factors to obtain desired cell types (e.g., T cells).

Different cell types can be obtained via appropriate isolation methods. The isolation methods include the separation of different cell types based on the expression or presence in the cell of one or more specific molecules, such as surface markers, e.g., surface proteins, intracellular markers, or nucleic acid. Any known method for separation based on such markers can be used. The separation may be affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. Both fractions may be retained for further use. Negative selection can be particularly useful where no antibody is available that specifically identifies a cell type in a heterogeneous population, such that separation is best carried out based on markers expressed by cells other than the desired population.

Also provided are methods, nucleic acids, compositions, and kits, for expressing the one or more CARs, and/or for producing the genetically engineered immune cells expressing the one or more CARs. The genetic engineering generally involves introduction of a nucleic acid encoding the one or more CARs into the cell, such as by retroviral transduction, transfection, or transformation. Gene transfer may be accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical application.

Recombinant nucleic acids may be transferred into cells using recombinant infectious virus particles, such as, e.g., vectors derived from simian virus 40 (SV40), adenoviruses, adeno-associated virus (AAV). Recombinant nucleic acids may be transferred into T cells using recombinant lentiviral vectors or retroviral vectors, such as gamma-retroviral vectors. The retroviral vector may have a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. The retroviruses may include those derived from any avian or mammalian cell source. The retroviruses typically are amphotropic, meaning that they are capable of infecting host cells of several species, including humans. The vector may be a lentivirus vector. In certain embodiments, the vector comprises any one of the nucleic acids encoding a CAR described herein. The nucleic acid can be cloned into the vector using any known molecular cloning methods in the art, including, for example, using restriction endonuclease sites and one or more selectable markers. Recombinant nucleic acids may be transferred into T cells via electroporation. Recombinant nucleic acids may be transferred into T cells via transposition. Other methods of introducing and expressing genetic material in immune cells include calcium phosphate transfection, protoplast fusion, cationic liposome-mediated transfection; tungsten particle-facilitated microparticle bombardment and strontium phosphate DNA co-precipitation. Many of these methods are descried e.g., in WO2019195486, which is incorporated herein by reference in its entirety. A T cells may be pre-activated, e.g., using anti-CD3/CD28 particles, for about 12 hours, about 24 hours, about 36 hours, about 48 hours, or about 60 hours prior to transduction. A transduced T cells may be harvested on day 5, day 6, day 7, day 8, day 9, day 10, day 11, or day 12 post transduction.

One of more CARs of the present disclosure may be expressed in at least 17%, for example 28%, of a population of cells which are engineered to express one or more of the CARs as measured by flow cytometry.

Transfection efficiency can be determined at 4 days post infection by an anti-VHH antibody via flow cytometry (e.g., following the methods described in Example 2). The transfection efficiency of the vectors may be at least 10%, at least 15%, at least 20%, at least 25%, at least 30%, at least 35%, at least 40%, at least 45%, at least 50%, at least 55%, at least 60%, at least 65%, at least 70%, at least 75%, or at least 80%.

CAR expression can be determined at 4 days post infection by an anti-VHH antibody via flow cytometry (e.g., following the methods described in Example 2). The CAR positive rate of the engineered immune cells may be at least 10%, at least 15%, at least 20%, at least 25%, at least 30%, at least 35%, at least 40%, at least 45%, at least 50%, or at least 55%.

The populations of engineered immune cells, compositions containing such cells and/or enriched for such cells, such as in which cells expressing the CAR may make up at least 15%, 20%, 25%, 30%, 35%, 40%, 50%, 60%, 70%, 80%, 90%, 91%, 92%, 93%, 94%, 95%, 96%, 97%, 98%, 99%, or more percent of the total cells in the composition or cells of a certain type such as T cells, CD8+ or CD4+ cells.

The viability of the transduced T cells may be at least 85%, at least 86%, at least 87%, at least 88%, at least 89%, at least 90%, at least 91%, at least 92%, at least 93%, at least 94%, or at least 95% on day 0, day 1, day 2, day 3, day 4, or day 5 post transduction. The viability of the transduced T cells may be at least or about 80%, at least or about 90%, at least or about 100%, at least or about 110%, at least or about 120% as compared to the viability of un-transduced T cells, on day 0, day 1, day 2, day 3, day 4, or day 5 (e.g., on day 5) post transduction.

The T cell expansion fold may be at least 1 fold, 2 folds, 3 folds, 4 folds, 5 folds, 10 folds, 15 folds, 20 folds, 25 folds, 30 folds, 35 folds, 40 folds, 45 folds, or 50 folds, on day 0, day 1, day 2, day 3, day 4, or day 5 post transduction. The T cell expansion fold of the transduced T cells may be at least or about 50%, at least or about 60%, at least or about 70%, at least or about 80%, at least or about 90% as compared to that of un-transduced T cells, on day 0, day 1, day 2, day 3, day 4, or day 5 (e.g., on day 5) post transduction.

The in vitro cytotoxicity of the engineered immune cells may be determined using an LDH (lactate dehydrogenase) based cytotoxicity assay (e.g., following the methods described in Example 3). The engineered immune cells (e.g., CAR-T cells) may be co-cultured with target cells for at least or about 1 hour, 2 hours, 3 hours, 4 hours, 5 hours, 6 hours, 12 hours, 16 hours, 18 hours, 1 day, 2 days, 3 days, or longer, such that the engineered immune cells (e.g., CAR-T cells) can be activated. The engineered immune cells may be cultured with different tumor cell lines (e.g., DLL3-positive SHP-77 cells, SHP-77-Luc-DLL3 KO or NK-92 cells) for 24 hours. The E:T ratio may be 0.5:1 or 2:1.

The in vitro cytotoxicity of the engineered immune cells may be at least 15%, at least 20%, at least 30%, at least 40%, at least 50%, at least 60%, at least 70%, at least 80%, at least 90%, or at least 95%, e.g., using the method as described in the Examples. The in vitro cytotoxicity of the engineered immune cells may be below 15%, below 20%, below 30%, below 40%, below 50%, below 60%, below 70%, below 80%, below 90%, below 95%. The in vitro cytotoxicity of the engineered immune cells may be about 40%-90%, about 40%-80%, or about 50%-80%. The cytotoxicity may specific for the target cells that express the first or the second antigen, for example a neuroendocrine lineage marker, for example DLL3, CD56, MUCIC, CDH17, GD2, Netrin3, SEZ6, GPC2 and/or B7-H3, for example DLL3 and CD56. The engineered immune cells may induce little or background cytotoxicity against target cells which do not express the first or the second antigen, for example do not express a neuroendocrine lineage marker, for example do not express DLL3, CD56, MUCIC, CDH17, GD2, Netrin3, SEZ6, GPC2 and/or B7-H3, for example do not express DLL3 and CD56.

Engineered immune cells comprising the dual-CAR system may have in vitro cytotoxicity, as measured by the method described in the Examples, which is higher than engineered immune cells comprising only one CAR comprising a primary intracellular signaling domain, when the engineered immune cells are exposed to target cells comprising a target antigen, for example at least 30% higher, for example at least 60% higher. Beneficially higher cytotoxicity may lead to more target tumor cells being killed.

Engineered immune cells comprising the dual-CAR system wherein the second CAR comprises a mutant hinge domain and mutant transmembrane domain, for example a mutant CD28 hinge domain, and a mutant CD28 transmembrane domain, for example a hinge domain comprising the amino acid sequence of SEQ ID NO: 33 and a transmembrane domain comprising the amino acid sequence of SEQ ID NO: 36, may have lower cytotoxicity as measured according to the method of the Examples compared to engineered immune cells comprising the dual-CAR system comprising a wild type hinge domain and transmembrane domain when the engineered immune cells are exposed to target cells comprising a target antigen, for example at least 15% lower. It may be beneficial to lower the cytotoxicity of the engineered immune cells as this may reduce any risk associated with overactivation of a dual-CAR system, for example it may reduce the risk of a cytokine storm by aiding control of immune cell activation.

The long-term cytotoxicity of the engineered immune cells (e.g., CAR-T cells) is determined, e.g., by re-challenging the engineered immune cells. Exemplary re-challenging procedures of CAR-T cells can be found, e.g., in Wang, Dongrui, et al., Journal of Visualized Experiments: JoVE 144 (2019); Wang D, et al., JCI Insight 2018, 3 (10); Lange et al., Cancer Discov. 2021 Feb. 9, candisc.0896.2020; each of which is incorporated herein by reference in its entirety.

The engineered immune cells can secrete cytokines (e.g., IFNγ), with or without exposure to antigens to which the antigen binding domains recognize. Cytokine release can be measured in an in vitro cytokine release assay (e.g., following the methods described in Example 4). Concentration of the cytokines (e.g., IFN-γ) released by the engineered immune cells (e.g., CAR-T cells) described herein can be determined by homogeneous time resolved fluorescence (HTRF) assays. The engineered immune cells may be co-cultured with tumor cells. The effector cell:target cell (E:T) ratio may be 0.5:1 or 2:1. The engineered immune cells may secrete an amount of IFNγ that is more than 50 pg/ml, more than 100 pg/ml, more than 200 pg/ml, more than 300 pg/ml, more than 400 pg/ml, more than 500 pg/ml, more than 1000 pg/ml, more than 1500 pg/ml, more than 2000 pg/ml, more than 2500 pg/ml, or more than 3000 pg/ml. The engineered immune cells may secrete an amount of IFNγ that is less than 50 pg/ml, less than 100 pg/ml, less than 200 pg/ml, less than 300 pg/ml, less than 400 pg/ml, less than 500 pg/ml, less than 1000 pg/ml, less than 1500 pg/ml, less than 2000 pg/ml, less than 2500 pg/ml, or less than 3000 pg/ml. The engineered immune cells may secrete an amount of IFNγ that is 500-5000 pg/ml, 1000-4000 pg/ml, 1000-3000 pg/ml, 1500-3000 pg/ml, 50-500 pg/ml, 50-400 pg/ml, 100-1000 pg/ml, 100-800 pg/ml, 100-600 pg/ml, 100-400 pg/ml, 200-400 pg/ml, 200-300 pg/ml, 200-1000 pg/ml, or 200-800 pg/ml. The engineered immune cells comprising the dual CAR system may secrete more IFNγ comparing to the engineered immune cells containing a single CAR. The engineered immune cells may secrete more IFNγ comparing to un-transfected control cells. The engineered immune cells may have an increased cytokine (e.g., IFN-γ) expression or secretion compared to wild type, i.e. non-engineered cells. The cytokine release may increase by at least or about 1 fold, 2 folds, 3 folds, 4 folds, 5 folds, 10 folds, 20 folds, 30 folds, 40 folds, 50 folds, 60 folds, 70 folds, 80 folds, 90 folds, 100 folds, 500 folds, 1000 folds, 2000 folds, 3000 folds, 4000 folds, 5000 folds, or 10000 folds.

Engineered immune cells comprising the dual-CAR system may have in vitro IFNγ production, as measured by the method described in the Examples, which is higher than engineered immune cells comprising only one CAR comprising a primary intracellular signaling domain, when the engineered immune cells are exposed to target cells comprising a target antigen.

Engineered immune cells comprising the dual-CAR system may have in vitro IFNγ production of at least about 2000 pg/ml when exposed to target cells at an E:T ratio of 0.5:1 which comprise the first and second target antigens according to the method of the Examples.

Engineered immune cells comprising the dual-CAR system may have in vitro IFNγ production of less than about 1000 pg/ml when exposed to target cells (comprising one of the first antigen or the second antigen) at an E:T ratio of 2:1 according to the method of the Examples.

Engineered immune cells comprising the dual-CAR system may have in vitro IFNγ production of less than about 500 pg/ml when exposed to target cells (comprising one of the first antigen or the second antigen) at a ratio of 0.5:1 according to the method of the Examples.

Engineered immune cells comprising the dual-CAR system wherein the second CAR comprises a mutant hinge domain and mutant transmembrane domain, for example a mutant CD28 hinge domain, and a mutant CD28 transmembrane domain, for example a hinge domain comprising the amino acid sequence of SEQ ID NO: 33 and a transmembrane domain comprising the amino acid sequence of SEQ ID NO: 36, may have lower in vitro IFNγ production as measured according to the method of the Examples compared to dual-CARs comprising a wild type hinge domain and transmembrane domain when exposed to target cells which express the first and/or second antigen.

The engineered immune cells may be re-challenged for at least 1, 2, 3, 4, 5, or 6 times. In some embodiments, the calculated cytotoxicity (Cytotoxicity %) is determined after each re-challenge. In some embodiments, after the first re-challenged, the calculated cytotoxicity of the engineered immune cells described herein is at least 70%, at least 75%, at least 80%, at least 85%, at least 90%, or at least 95%. After the second re-challenge, the calculated cytotoxicity of the engineered immune cells may be at least 80%, at least 90%, or at least 95%. After the third re-challenge, the calculated cytotoxicity of the engineered immune cells may be at least 30%, at least 40%, at least 50%, at least 60%, at least 70%, at least 75%, at least 80%, at least 85%, at least 90%, or at least 95%. After the fourth re-challenge, the calculated cytotoxicity of the engineered immune cells may be at least 15%, at least 20%, at least 30%, at least 40%, at least 50%, at least 60%, at least 70%, at least 80%, at least 90%, or at least 95%. After the fifth re-challenge, the calculated cytotoxicity of the engineered immune cells may be at least 15%, at least 20%, at least 30%, at least 40%, at least 50%, at least 60%, at least 70%, at least 80%, at least 90%, or at least 95%. After the sixth re-challenge, the calculated cytotoxicity of the engineered immune cells may be at least 0%, at least 5%, at least 10%, at least 15%, at least 20%, at least 30%, at least 40%, at least 50%, at least 60%, at least 70%, at least 80%, at least 90%, or at least 95%. In some embodiments, the maximum re-challenge number (i.e., the number of re-challenge times before tumor cells outgrow) of the engineered immune cells described herein is at least 5 times, 6 times, 7 times, 8 times, 9 times, or 10 times.

The persistence and/or exhaustion of the engineered immune cells in vitro may be evaluated by repeatedly stimulating the engineered immune cells with tumor cells (e.g., SHP-77 cells) for several rounds in a re-challenge assay (e.g., following the methods described in Example 5). The engineered immune cells can be first challenged (co-cultured) with tumor cells (e.g., SHP-77 cells) for a first round, and then re-challenged with fresh tumor cells (e.g., SHP-77 cells) in a subsequent round. The E:T ratio may be 1:5. The percentage of CD3 positive rate in the total live cells may be more than 5%, more than 10%, more than 15%, more than 20%, more than 30%, more than 40%, more than 50%, more than 60%, more than 70%, more than 80%, more than 90%, or more than 95%, after 1 round, 2 rounds, 3 rounds, or 4 rounds of stimulation in a re-challenge assay. The total T cells may expand by more than 1 fold, more than 2 fold, more than 3 fold, more than 4 fold, more than 5 fold, more than 6 fold, more than 7 fold, more than 8 fold, more than 9 fold, more than 10 fold, more than 15 fold, more than 20 fold, more than 25 fold, more than 30 fold, more than 35 fold, more than 40 fold, more than 45 fold, more than 50 fold, more than 60 fold, more than 70 fold, more than 80 fold, more than 90 fold, more than 100 fold, more than 110 fold, more than 120 fold, more than 130 fold, more than 140 fold, more than 150 fold, more than 200 fold, or more than 250 fold, after 1 round, 2 rounds, 3 rounds, or 4 rounds of stimulation in a re-challenge assay. The engineered immune cells may expand by more than 1 fold, more than 2 fold, more than 3 fold, more than 4 fold, more than 5 fold, more than 6 fold, more than 7 fold, more than 8 fold, more than 9 fold, more than 10 fold, more than 15 fold, more than 20 fold, more than 25 fold, more than 30 fold, more than 35 fold, more than 40 fold, more than 45 fold, more than 50 fold, more than 60 fold, more than 70 fold, more than 80 fold, more than 90 fold, more than 100 fold, more than 110 fold, more than 120 fold, more than 130 fold, more than 140 fold, more than 150 fold, more than 200 fold, more than 250 fold, more than 500 fold, more than 1000 fold, more than 2000 fold, more than 3000 fold, or more than 4000 fold, after 1 round, 2 rounds, 3 rounds, or 4 rounds of stimulation in a re-challenge assay. Each round of stimulation may last for 1 day, 2 days, 3 days, 4 days or 5 days. In some embodiments, each round of stimulation lasts for 3 days. The engineered immune cells may exhibit better long-term persistence than wild type cells which are not engineered. The amount of engineered immune cells may increase, comparing to control cells, by more than 5%, more than 10%, more than 15%, more than 20%, more than 30%, more than 40%, more than 50%, more than 60%, more than 70%, more than 80%, more than 90%, more than 100%, more than 150%, more than 200%, more than 250%, more than 300%, more than 400%, more than 500%, more than 600%, more than 700%, more than 800%, more than 900%, or more than 10,00%. The engineered immune cell comprising the dual-CAR system may exhibit better long-term persistence than engineered immune cells comprising only a single CAR (e.g., the first CAR in the dual-CAR system). In some embodiments, comparing to the single CAR engineered immune cells, the amount of proliferation of the dual-CAR engineered immune cells increases by more than 5%, more than 10%, more than 15%, more than 20%, more than 30%, more than 40%, more than 50%, more than 60%, more than 70%, more than 80%, more than 90%, more than 100%, more than 150%, more than 200%, more than 250%, more than 300%, more than 400%, more than 500%, more than 600%, more than 700%, more than 800%, more than 900%, or more than 10,00%.

The in vivo tumor suppression by the engineered immune cells can be evaluated by an in vivo tumor suppression assay (e.g., following the methods described in Example 6). The tumor model can be constructed by subcutaneously injecting NCG mice with SCLC NCI-H82 cells. The engineered immune cells can be administered intravenously to tumor grafted mice 10 days after tumor inoculation. Tumor length (L) and width (W) can be measured by caliper every 3-4 days after CAR-T cells injection. Tumor volume can be estimated using formula: V=(W2×L)/2. The engineered immune cells may suppress tumor growth by more than 5%, more than 10%, more than 15%, more than 20%, more than 30%, more than 40%, more than 50%, more than 60%, more than 70%, more than 80%, more than 90%, more than 100%, more than 150%, more than 200%, more than 250%, more than 300%, more than 400%, more than 500%, more than 600%, more than 700%, more than 800%, more than 900%, or more than 10,00%, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, or 35 days. The body weights of the mice may not change significantly throughout the experiment. The body weights of the mice may change by less than 5%, less than 10%, less than 20%, less than 30%, less than 40%, less than 50%, less than 60%, less than 70%, less than 80%, less than 90%, or less than 100%, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, or 35 days. The engineered immune cells comprising the dual-CAR system may have more pronounced tumor suppression than the engineered immune cells comprising only a single CAR (e.g., the first CAR in the dual-CAR system), by more than 5%, more than 10%, more than 15%, more than 20%, more than 30%, more than 40%, more than 50%, more than 60%, more than 70%, more than 80%, more than 90%, more than 100%, more than 150%, more than 200%, more than 250%, more than 300%, more than 400%, more than 500%, more than 600%, more than 700%, more than 800%, more than 900%, or more than 10,00%, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, or 35 days.

The engineered immune cells comprising the dual-CAR system may increase the total immune cells (e.g., T cells) compared to an engineered immune cell comprising only a single CAR (e.g., the first CAR in the dual-CAR system), for example may increase the total immune cells (e.g., T cells) by more than 1 fold, more than 2 fold, more than 3 fold, more than 4 fold, more than 5 fold, more than 6 fold, more than 7 fold, more than 8 fold, more than 9 fold, more than 10 fold, more than 15 fold, more than 20 fold, more than 25 fold, more than 30 fold, more than 35 fold, more than 40 fold, more than 45 fold, or more than 50 fold, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, or 25 days. The engineered immune cells comprising the dual-CAR system may increase the number of immune cells (e.g., T cells) compared to an engineered immune cell comprising only one single CAR (e.g., the first CAR in the dual-CAR system), for example may increase the immune cells (e.g., T cells) by more than 1 fold, more than 2 fold, more than 3 fold, more than 4 fold, more than 5 fold, more than 6 fold, more than 7 fold, more than 8 fold, more than 9 fold, more than 10 fold, more than 15 fold, more than 20 fold, more than 25 fold, more than 30 fold, more than 35 fold, more than 40 fold, more than 45 fold, more than 50 fold, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, or 25 days. The engineered immune cells may exhibit better long-term persistence compared to the un-transduced immune cells, i.e. immune cells which do not comprise a CAR. In some embodiments, the proliferation rate of the engineered immune cells may be higher than un-transduced immune cells (e.g., immune cells which do not comprise a CAR), by more than 5%, more than 10%, more than 15%, more than 20%, more than 30%, more than 40%, more than 50%, more than 60%, more than 70%, more than 80%, more than 90%, more than 100%, more than 150%, more than 200%, more than 250%, more than 300%, more than 400%, more than 500%, more than 600%, more than 700%, more than 800%, more than 900%, or more than 10,00%, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, or 25 days. Engineered immune cells comprising the dual-CAR system exhibit better long-term persistence compared to engineered immune cells comprising only a single CAR (e.g., the first CAR in the dual-CAR system). In some embodiments, the proliferation rate of the engineered immune cells may be higher than engineered immune cells comprising only a single CAR (e.g., the first CAR in the dual-CAR system), by more than 5%, more than 10%, more than 15%, more than 20%, more than 30%, more than 40%, more than 50%, more than 60%, more than 70%, more than 80%, more than 90%, more than 100%, more than 150%, more than 200%, more than 250%, more than 300%, more than 400%, more than 500%, more than 600%, more than 700%, more than 800%, more than 900%, or more than 10,00%, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, or 25 days.

The present disclosure provides a method or process for preparing, manufacturing and/or using the engineered immune cells for treatment of pathological diseases or conditions.

The cells for engineering can be isolated from a sample, such as a biological sample, e.g., one obtained from or derived from a subject. In some embodiments, the subject from which the cell is isolated is one having the disease or condition or in need of a cell therapy or to which cell therapy will be administered. The subject may be a human in need of a particular therapeutic intervention, such as the adoptive cell therapy for which cells are being isolated, processed, and/or engineered.

The cells may be primary cells, e.g., primary human cells. The samples may include tissue, fluid, and other samples taken directly from the subject, as well as samples resulting from one or more processing steps, such as separation, centrifugation, genetic engineering (e.g., transduction with viral vector), washing, and/or incubation. The biological sample may be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

### 3. Nucleic Acids

In one aspect, the present disclosure provides (i) nucleic acids encoding the dual-CARs described herein, and/or (ii) nucleic acids encoding the single CARs described herein.

In one aspect, the present disclosure provides a nucleic acid comprising one or more nucleic acid sequences that encode a first chimeric antigen receptor (CAR) and a second chimeric antigen receptor (CAR), wherein
(a) the first chimeric antigen receptor (CAR) comprising:
i) an antigen binding domain specifically recognizing a first antigen;
ii) a first transmembrane domain; and
iii) a first intracellular signaling domain comprising a co-stimulatory signaling domain of the first intracellular signaling domain; and
(b) the second chimeric antigen receptor (CAR) comprising:
i) a second antigen binding domain specifically recognizing a second antigen;
ii) a second transmembrane domain; and
iii) a second intracellular signaling domain comprising a co-stimulatory signaling domain of the second intracellular signaling domain, wherein the first intracellular signaling domain further comprises a first primary intracellular signaling domain of an immune cell, and the second intracellular signaling domain does not comprise a primary intracellular signaling domain that is derived from CD3ζ; and wherein the first antigen and the second antigen are selected from the group consisting of DLL3, CD56, MUCIC, CDH17, GD2, Netrin3, SEZ6, GPC2 and B7-H3. In some embodiments, the second intracellular signaling domain does not comprise a primary intracellular signaling domain of immune cell.

In some embodiments, the nucleic acid may comprise one or more nucleic acid sequences that encode a first chimeric antigen receptor (CAR) and a second chimeric antigen receptor (CAR). In some embodiments, the first chimeric antigen receptor (CAR) may be a DLL3 CAR comprising: (a) a first antigen binding domain comprising an anti-DLL3 binding moiety comprising two anti-DLL3 sdAbs (e.g., VHH domains); (b) a first transmembrane domain; and (c) a first intracellular signaling domain, wherein the two anti-DLL3 sdAbs comprises any one of the following: (1) a first anti-DLL3 sdAb comprising a CDR1 comprising the amino acid sequence of SEQ ID NO: 7, a CDR2 comprising the amino acid sequence of SEQ ID NO: 8, and a CDR3 comprising the amino acid sequence of SEQ ID NO: 9; and a second anti-DLL3 sdAb comprising a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12, wherein the CDRs are defined according to the Kabat numbering scheme; or (2) a first anti-DLL3 sdAb comprising a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21; and a second anti-DLL3 sdAb comprising a CDR1 comprising the amino acid sequence of SEQ ID NO: 22, a CDR2 comprising the amino acid sequence of SEQ ID NO: 23, and a CDR3 comprising the amino acid sequence of SEQ ID NO: 24, wherein the CDRs are defined according to the AbM numbering scheme. The anti-DLL3 sdAb may be camelid, chimeric, human, or humanized. The VHH CDRs (CDR1-3) may be determined according to the Kabat numbering scheme, the IMGT numbering scheme, the AbM numbering scheme, the Chothia numbering scheme, the Contact numbering scheme, or a combination thereof. In some embodiments, the anti-DLL3 sdAb comprises a VHH domain comprising an amino acid sequence selected from the group consisting of SEQ ID NOs: 28-29. In some embodiments, the anti-DLL3 binding moiety comprises a first VHH domain comprising the amino acid sequence of SEQ ID NO: 28, or at least 80%, 85%, 90%, or 95% identical to the amino acid sequence set forth in SEQ ID NO: 28; and a second VHH domain comprising the amino acid sequence of SEQ ID NO: 29, or at least 80%, 85%, 90%, or 95% identical to the amino acid sequence set forth in SEQ ID NO: 29. In some embodiments, the first intracellular signaling domain comprises a co-stimulatory signaling domain and a primary intracellular signaling domain of an immune effector cell (such as T cell). In some embodiments, the primary intracellular signaling domain is derived from CD35. The co-stimulatory signaling domain of the first intracellular signaling domain may be derived from a molecule selected from the group consisting of CD27, CD28, CD137, OX40, CD30, CD40, CD3, LFA-1, ICOS, CD2, CD7, LIGHT, NKG2C, B7-H3, and ligands of CD83. In some embodiments, the DLL3 CAR further comprises a hinge domain (such as a CD8a hinge domain) located between the C-terminus of the DLL3 antigen binding domain and the N-terminus of the transmembrane domain. In some embodiments, the DLL3 CAR further comprises a signal peptide (such as a CD8a signal peptide) located at the N-terminus of the DLL3 antigen binding domain. In some embodiments, the first CAR includes the polypeptide comprising from the N-terminus to the C-terminus: a CD8a signal peptide, the DLL3 antigen-binding domain, a CD8a hinge domain, a CD8a transmembrane domain, a co-stimulatory signaling domain derived from 4-1BB, and a primary intracellular signaling domain derived from CD35. In some embodiments, the second CAR may be CD56 CAR comprising: (a) a second antigen binding domain comprising an anti-CD56 binding moiety comprising an anti-CD56 scFv (e.g., VL-VH pairs); (b) a second transmembrane domain; and (c) a second intracellular signaling domain comprising a co-stimulatory signaling domain, wherein the second intracellular signaling domain does not comprise a primary intracellular signaling domain that is derived from CD34, and wherein the anti-CD56 scFv comprises any one of the following: (a) a VH domain comprising a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; and a VL domain comprising a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, wherein the CDRs are defined according to the Kabat numbering scheme; or (b) a VH domain comprising a HCDR1 comprising the amino acid sequence of SEQ ID NO: 13, a HCDR2 comprising the amino acid sequence of SEQ ID NO: 14, and a HCDR3 comprising the amino acid sequence of SEQ ID NO: 15; and a VL domain comprising a LCDR1 comprising the amino acid sequence of SEQ ID NO: 16, a LCDR2 comprising the amino acid sequence of SEQ ID NO: 17, and a LCDR3 comprising the amino acid sequence of SEQ ID NO: 18, wherein the CDRs are defined according to the AbM numbering scheme. The anti-CD56 scFv may be mouse, chimeric, human, or humanized. The CDRs of anti-CD56 scFv e.g., VH CDRs (HCDR1-3) and VL CDRs (LCDR1-3) may be determined according to the Kabat numbering scheme, the IMGT numbering scheme, the AbM numbering scheme, the Chothia numbering scheme, the Contact numbering scheme, or a combination thereof. In some embodiments, the anti-CD56 scFv comprises a VH domain comprising the amino acid sequence of SEQ ID NO: 25, or at least 80%, 85%, 90%, or 95% identical to the amino acid sequence set forth in SEQ ID NO: 25, and a VL domain comprising the amino acid sequence of SEQ ID NO: 26, or at least 80%, 85%, 90%, or 95% identical to the amino acid sequence set forth in SEQ ID NO: 26. In some embodiments, the second intracellular signaling domain does not comprise a primary intracellular signaling domain of an immune effector cell (such as T cell). The co-stimulatory signaling domain of the second intracellular signaling domain may be derived from a molecule selected from the group consisting of CD27, CD28, CD137, OX40, CD30, CD40, CD3, LFA-1, ICOS, CD2, CD7, LIGHT, NKG2C, B7-H3, and ligands of CD83. In some embodiments, the CD56 CAR further comprises a hinge domain (such as a CD28 hinge domain or a mutant CD28 hinge domain) located between the C-terminus of the CD56 antigen binding domain and the N-terminus of the second transmembrane domain. In some embodiments, the CD56 CAR further comprises a signal peptide (such as a CD8α signal peptide) located at the N-terminus of the CD56 antigen binding domain. In some embodiments, the first CAR includes the polypeptide comprises from the N-terminus to the C-terminus: a CD8α signal peptide, the CD56 antigen binding domain, a CD28 hinge domain or a mutant CD28 hinge domain, a transmembrane domain derived from CD28, and a co-stimulatory signaling domain derived from CD28.

A nucleic acid of the present disclosure may comprise a first nucleic acid sequence and a second nucleic acid sequence. The first nucleic acid may be upstream of the second nucleic acid or the first nucleic acid may be downstream of the second nucleic acid. The first and second nucleic acid sequence can be separated by a linker. A linker for use in the present disclosure allows for multiple proteins to be encoded by the same nucleic acid sequence (e.g., a multicistronic or bicistronic sequence), which are translated as a polyprotein that is dissociated into separate protein components. The nucleic acid may comprise from 5′ end to 3′ end of the first nucleic acid sequence, the linker, and the second nucleic acid sequence. The nucleic acid may comprise from 5′ end to 3′ end the second nucleic acid sequence, the linker, and the first nucleic acid sequence. The first nucleic acid sequence may encode a first CAR described herein and the second nucleic acid sequence may encode a second CAR described herein.

The linker may comprise a nucleic acid sequence that encodes for an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunogloublin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES.

The linker may comprise a nucleic acid sequence that encodes for a self-cleaving peptide. As used herein, a “self-cleaving peptide” or “2A linker” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A linkers are known to those of skill in the art, including, without limitation, those found in members of the Picornaviridae virus family, e.g., foot-and-mouth disease virus (FMDV), equine rhinitis A virus (ERAVO, Thosea asigna virus (TaV), and porcine tescho virus-1 (PTV-1); and carioviruses such as Theilovirus and encephalomyocarditis viruses. 2A linkers derived from FMDV, ERAV, PTV-1, and TaV are referred to herein as “F2A,” “E2A,” “P2A,” and “T2A,” respectively. The P2A linker may have a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 45. The T2A linker may have a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 46.

Various linker sequences are known in the art, including, without limitation, glycine serine (GS) spacers (also known as GS linkers) such as (GS)n, (SG)n, (GSGGS)n and (GGGS)n, where n represents an integer of at least 1. Exemplary linker sequences can comprise amino acid sequences including, without limitation, GGGGSGGGGSGGGGS (SEQ ID NO: 47), GGSG (SEQ ID NO: 48), GGSGG (SEQ ID NO: 49), GSGSG (SEQ ID NO: 50), GSGGG (SEQ ID NO: 51), GGGSG (SEQ ID NO: 52), GSSSG (SEQ ID NO: 53), and the like. Those of skill in the art would be able to select the appropriate linker sequence.

In some embodiments, the present disclosure provides a nucleic acid comprising one nucleic acid sequence that encodes a single CAR (e.g., CD56 CAR) comprising: (a) an antigen binding domain comprising an anti-CD56 binding moiety comprising an anti-CD56 scFv (e.g., VL-VH pairs); (b) a transmembrane domain; and (c) an intracellular signaling domain, wherein the anti-CD56 scFv comprises any one of the following: (a) a VH domain comprising a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3; and a VL domain comprising a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, wherein the CDRs are defined according to the Kabat numbering scheme; or (b) a VH domain comprising a HCDR1 comprising the amino acid sequence of SEQ ID NO: 13, a HCDR2 comprising the amino acid sequence of SEQ ID NO: 14, and a HCDR3 comprising the amino acid sequence of SEQ ID NO: 15; and a VL domain comprising a LCDR1 comprising the amino acid sequence of SEQ ID NO: 16, a LCDR2 comprising the amino acid sequence of SEQ ID NO: 17, and a LCDR3 comprising the amino acid sequence of SEQ ID NO: 18, wherein the CDRs are defined according to the AbM numbering scheme. In some embodiments, the anti-CD56 scFv comprises a VH domain comprising the amino acid sequence of SEQ ID NO: 25, or at least 80%, 85%, 90%, or 95% identical to the amino acid sequence set forth in SEQ ID NO: 25, and a VL domain comprising the amino acid sequence of SEQ ID NO: 26, or at least 80%, 85%, 90%, or 95% identical to the amino acid sequence set forth in SEQ ID NO: 26. The intracellular signaling domain may comprise a co-stimulatory signaling domain and a primary intracellular signaling domain of an immune effector cell (such as T cell). In some embodiments, the intracellular signaling domain may not comprise a primary intracellular signaling domain of an immune effector cell (such as T cell). The co-stimulatory signaling domain of the intracellular signaling domain may be derived from a molecule selected from the group consisting of CD27, CD28, CD137 (4-1BB), OX40, CD30, CD40, CD3, LFA-1, ICOS, CD2, CD7, LIGHT, NKG2C, B7-H3, and ligands of CD83. In some embodiments, the CD56 CAR further comprises a hinge domain (such as a CD28 hinge domain or mutant CD28 hinge domain) located between the C-terminus of the CD56 antigen binding domain and the N-terminus of the transmembrane domain. In some embodiments, the CD56 CAR further comprises a signal peptide (such as a CD8α signal peptide) located at the N-terminus of the CD56 antigen binding domain. In some embodiments, the CD56 CAR includes the polypeptide comprising from the N-terminus to the C-terminus: a CD8α signal peptide, the CD56 antigen binding domain, a CD28 hinge domain or a mutant CD28 hinge domain, a transmembrane domain derived from CD28, and a co-stimulatory signaling domain derived from CD28.

A nucleic acid of the present disclosure may a restriction enzyme site sequence.

A nucleic acid of the present disclosure can be operably linked to a transcriptional control element, e.g., a promoter, and enhancer, etc.

The promoter may be a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

Other examples of suitable promoters include the immediate early cytomegalovirus (CMV) promoter sequence. This promoter sequence is a strong constitutive promoter sequence capable of driving high levels of expression of any nucleic acid sequence operatively linked thereto. Other constitutive promoter sequences can also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the disclosure should not be limited to the use of constitutive promoters. Inducible promoters are also contemplated as part of the disclosure. The use of an inducible promoter provides a molecular switch capable of turning on expression of the nucleic acid sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The nucleic acid of the present disclosure may be provided for the production of a CAR of a dual-CAR system (e.g., in a mammalian cell). The nucleic acid of the present disclosure may provide for amplification of the nucleic acid.

A vector, for example an expression vector (e.g., a lentiviral vector) can be used to introduce the nucleic acid encoding for the dual-CARs or the single CAR into an immune cell (e.g., a T cell) or precursor thereof. The vector (e.g., a lentiviral vector) of the present disclosure may comprise one or more nucleic acids encoding for the dual-CAR of the present disclosure. The vector (e.g., a lentiviral vector) of the present disclosure may comprise one nucleic acid encoding for the single CAR of the present disclosure. The vector (e.g., lentiviral vector) may comprise additional elements that will aid in the functional expression of the CARs encoded therein. The expression vector comprising nucleic acids encoding for a dual-CAR or single CAR may comprise a mammalian promoter. The vector may comprise an elongation-factor-1-alpha promoter (EF-1α promoter). The use of an EF-1α promoter may increase the efficiency in expression of downstream transgenes (e.g., a CAR encoding nucleic acid). Physiologic promoters (e.g., an EF-1α promoter) may be less likely to induce integration mediated genotoxicity, and may abrogate the ability of the retroviral vector to transform stem cells. Other physiological promoters suitable for use in a vector (e.g., lentiviral vector) are known to those of skill in the art and can be incorporated into a vector of the present disclosure. The vector (e.g., lentiviral vector) may comprise a non-requisite cis acting sequence that can improve titers and gene expression.

A nucleic acid may encode the first CAR (in the dual-CAR system) comprising from the N-terminus to the C-terminus, a CD8α signal peptide, an antigen binding domain, a CD8α hinge domain, a CD8α transmembrane domain, a 4-1BB co-stimulatory signaling domain, and a CD3 ζ primary intracellular signaling domain. The nucleic acid may encode a DLL3 CAR. The nucleic acid may encode an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 40.

A nucleic acid may encode the second CAR (in the dual-CAR system) or the CD56 CAR comprising from the N-terminus to the C-terminus, a CD8α signal peptide, an antigen binding domain, a CD28 hinge domain or a mutant CD28 hinge domain, a CD28 transmembrane domain or a mutant CD28 transmembrane domain, and a CD28 co-stimulatory signaling domain. In some embodiments, the nucleic acid encodes a CD56 CAR. In some embodiments, the nucleic acid encodes an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41 or SEQ ID NO: 42.

In some embodiments, the nucleic acid encodes a dual-CAR system comprising a DLL3 CAR and a CD56 CAR. In some embodiments, the nucleic acid comprises from the N-terminus to the C-terminus, the coding sequences of a first CD8α signal peptide, a first antigen binding domain targeting DLL3, a CD8α hinge domain, a CD8α transmembrane domain, a 4-1BB co-stimulatory signaling domain, a CD3ζ primary intracellular signaling domain, a 2A linker, a second CD8α signal peptide, a second antigen binding domain targeting CD56, a CD28 hinge domain (wild-type or mutant), a CD28 transmembrane domain (wild-type or mutant), and a CD28 co-stimulatory signaling domain. In some embodiments, the nucleic acid encodes an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 43 or SEQ ID NO: 44.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

### 4. Methods of Treatment

The CAR, the dual CAR system, the nucleic acids and the engineered immune cells of the disclosure can be used in a variety of experimental, therapeutic and commercial applications.

In one aspect, the disclosure provides a method of modulating an immune response comprising administering an effective amount of engineered immune cells described herein to a subject in need thereof.

The term “effective amount” as used herein means an amount effective, at dosages and for periods of time necessary to achieve the desired results.

In another aspect, the present disclosure provides a method for treating cancer comprising administering an effective amount of engineered immune cells described herein to a subject in need thereof.

In one aspect, the engineered immune cells described herein may be for use in the treatment of a subject having cancer of being at risk of having cancer.

The subject may have a neuroendocrine neoplasm (NENs), for example a neuroendocrine tumor (NET), for example a neuroendocrine carcinoma (NEC).

Examples of cancer that can be treated include, but are not limited to, small cell lung cancer (SCLC), large cell neuroendocrine cancer (LCNC), neuroendocrine prostate cancer (NEPC), pancreatic neuroendocrine tumor (PNET) and gastrointestinal neuroendocrine cancers.

The disclosure further includes the use of the engineered immune cells described herein in the manufacture of a medicament or pharmaceutical composition to modulate an immune response or to treat cancer as described hereinabove.

The engineered immune cells can also be used in experimental models, for example, to further study and elucidate the function of the cells.

One or more of the engineered immune cells described herein can be administered to a subject in a single, unified form, such as an intravenous injection, or in multiple forms, for example, as multiple intravenous infusions or injections, or subcutaneous injections. In some cases, the engineered immune cells can expand within a subject's body, in vivo, after administration to a subject. The engineered immune cells can be frozen to provide cells for multiple treatments with the same cell preparation. The engineered immune cells of the disclosure, and pharmaceutical compositions comprising the same, can be packaged as a kit. A kit can include instructions (e.g., written instructions) on the use of the engineered immune cells and compositions comprising the same.

Methods for administration of engineered immune cells for adoptive cell therapy are known and can be used in connection with the provided methods and compositions. For example, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., a patient, in need of a treatment and the cells, following isolation and processing are administered to the same subject.

The cell therapy (e.g., adoptive T cell therapy), may be carried out by allogeneic transfer, in which the cells are isolated and/or otherwise prepared from a subject other than a subject who is to receive or who ultimately receives the cell therapy, e.g., a first subject, the cells then are administered to a different subject, e.g., a second subject, of the same species. The first and second subjects may be genetically identical. The first and second subjects may be genetically similar. The second subject may express the same HLA class or supertype as the first subject.

The engineered immune cells described herein may be administered to an animal, for example a mammal, even a human, to treat a cancer. In addition, the engineered immune cells may be used for the treatment of any condition related to a cancer, especially a cell-mediated immune response against a tumor cell(s), where it is desirable to treat or alleviate the disease. The types of cancers to be treated with the engineered immune cells or pharmaceutical compositions include, but are not limited to neuroendocrine neoplasm (NEN), for example a neuroendocrine tumor (NET), for example a neuroendocrine carcinoma (NEC), small cell lung cancer (SCLC), large cell neuroendocrine cancer (LCNC), neuroendocrine prostate cancer (NEPC), pancreatic neuroendocrine tumor (PNET) and gastrointestinal neuroendocrine cancers.

The engineered immune cells (e.g., T cells, or NK cells) described herein can be included in a composition for immunotherapy. The composition may include a pharmaceutical composition and further include a pharmaceutically acceptable carrier. A therapeutically effective amount of the pharmaceutical composition comprising the engineered immune cells may be administered.

The engineered immune cells may be immediately used in the above therapeutic, experimental or commercial applications or the cells can be cryopreserved for use at a later date. The pharmaceutical compositions can be included in a container, pack, or dispenser together with instructions for administration.

Any references to methods of treatment refers to the compounds, pharmaceutical compositions, and/or medicaments of the present disclosure for use as a medicament, for use in a method of treatment of an animal, for example a human, or for use in the treatment of disease, for example for use in the treatment of cancer.

### EXAMPLES

The disclosure is further described in the following examples, which do not limit the scope of the disclosure described in the claims.

### Example 1: Plasmid Construction, Virus Preparation, and Titer Evaluation

Briefly, the lentiviral vector PLVX-EFIA is created using pLVX-Puro (Clontech #632164), by replacing the original promoter with human elongation factor 1α promoter (hEF1α) and by removing the puromycin resistance gene with EcoRI and BamHI by GenScript. PLVX-EF1A is further subjected to the lentivirus packaging procedure as described below.

To generate viral particles comprising nucleic acids encoding the CAR constructs disclosed herein, lentivirus packaging plasmid mixture including pMDLg/pRRE (Addgene #11251), pRSV-Rev (Addgene #11253), and pMD2.G (Addgene #11259) are pre-mixed with a PLVX-EF1A (including target system) vector at a pre-optimized ratio with polyetherimide (PEI), mixed properly, and incubated at room temperature for 5 min. The transfection mix is added dropwise to 293-T cells and mixed gently. Transfected 293-T cells are incubated overnight at 37° C. and 5% CO₂. Twenty-four hours post-transfection, supernatants are collected and centrifuged at 4° C., 500 g for 10 min to remove any cellular debris. Centrifuged supernatants are filtered through a 0.45 μm PES filter to remove cell debris before ultracentrifugation. After ultracentrifugation, the supernatants are carefully discarded and the virus pellets are rinsed with pre-chilled DPBS. The concentration of virus is measured. Virus is aliquoted and stored at −80° C. Viral titers are determined by functional transduction on a T cell line.

### Example 2: Preparation of Engineered Immune Cells Expressing Chimeric Antigen Receptors

The constructs of DLL3 bispecific CAR (DLL3 CAR, SEQ ID NO: 40) and DLL3/CD56 dual-specific CARs (DLL3/CD56 Dual-CAR-1 #, SEQ ID NO: 43 and DLL3/CD56 Dual-CAR-2 #, SEQ ID NO: 44) were designed as shown in FIGS. 1A-1C. CAR backbone nucleotide sequences encoding DLL3 CAR and DLL3/CD56 Dual-CAR polypeptides were chemically synthesized and cloned into a pre-modified lentiviral vector (PLVX-EF1A).

Specifically, DLL3 CAR comprises a polypeptide from the N-terminus to the C-terminus: a CD8α signal peptide (SEQ ID NO: 30), an antigen binding domain having two anti-DLL3 V_{H}H domains (SEQ ID NOs: 28-29), a CD8α hinge domain (SEQ ID NO: 31), a CD8α transmembrane domain (SEQ ID NO: 34), a 4-1BB co-stimulatory signaling domain (SEQ ID NO: 37), and CD3ζ primary intracellular signaling domain (SEQ ID NO: 39).

DLL3/CD56 Dual-CAR comprises a nucleotide encoding a DLL3 CAR and a CD56 CAR. The nucleotide encoding the DLL3 CAR and the nucleotide encoding the CD56 CAR were connected by a nucleotide encoding a 2A linker. The 2A linker has the amino acid sequence set forth in SEQ ID NO: 46. The CD56 CAR (in the DLL3/CD56 Dual-CAR) may comprise a polypeptide from the N-terminus to the C-terminus: a CD8α signal peptide (SEQ ID NO: 30), an antigen binding domain having an anti-CD56 scFv (e.g., VL-VH domain, SEQ ID NO: 27), a CD28 hinge domain (SEQ ID NO: 32) or a mutant CD28 hinge domain (SEQ ID NO: 33), a CD28 transmembrane domain (SEQ ID NO: 35) or a mutant CD28 transmembrane domain (SEQ ID NO: 36), and a CD28 co-stimulatory signaling domain (SEQ ID NO: 38). CD56 CAR-1 #comprises a polypeptide having the amino acid sequence of SEQ ID NO: 41, and CD56 CAR-2 #comprises a polypeptide having the amino acid sequence of SEQ ID NO: 42.

TABLE 2
Exemplary CDRs According to Kabat and AbM Numbering Schemes

| Loop | Anti-CD56 VH | Anti-CD56 VL | Anti-DLL3 VHH1 | Anti-DLL3 VHH2 |
|---|---|---|---|---|
| Kabat | CDR1 (SEQ ID NO: 1) | CDR1 (SEQ ID NO: 4) | CDR1 (SEQ ID NO: 7) | CDR1 (SEQ ID NO: 10) |
| | CDR2 (SEQ ID NO: 2) | CDR2 (SEQ ID NO: 5) | CDR2 (SEQ ID NO: 8) | CDR2 (SEQ ID NO: 11) |
| | CDR3 (SEQ ID NO: 3) | CDR3 (SEQ ID NO: 6) | CDR3 (SEQ ID NO: 9) | CDR3 (SEQ ID NO: 12) |
| AbM | CDR1 (SEQ ID NO: 13) | CDR1 (SEQ ID NO: 16) | CDR1 (SEQ ID NO: 19) | CDR1 (SEQ ID NO: 22) |
| | CDR2 (SEQ ID NO: 14) | CDR2 (SEQ ID NO: 17) | CDR2 (SEQ ID NO: 20) | CDR2 (SEQ ID NO: 23) |
| | CDR3 (SEQ ID NO: 15) | CDR3 (SEQ ID NO: 18) | CDR3 (SEQ ID NO: 21) | CDR3 (SEQ ID NO: 24) |

Immune cells, in this case T cells, were isolated from healthy donor PBMCs (HemaCare) using a pan T cell isolation kit (Miltenyi Biotec, 130096535). Isolated T cells were cultured using AIMV (Gibco, 31035025) medium with 5% FBS (Gibco, 10099141) and further activated by CD3/CD28 activation beads (Miltenyi Biotec, 130091442) at a 2:1 ratio at 37° C. and 5% CO₂. 24 h or 72 h after initial activation, T cells were transduced with lentiviruses expressing a DLL3 targeting CAR at proper multiplicity of infection (MOI) in the presence of 8 μg/mL polybrene (SIGMA-ALDRICH, H9268-10G). Additional IL-2 was supplemented to a final concentration of 300 IU/mL. Fresh medium was replaced 24 h post lentiviral infection. Infected T cells were maintained in AIMV medium with 5% FBS and 300 IU/mL IL-2 at a cell density between 5×10⁵ to 1×10⁶ cells/mL.

CAR expression was determined at 4 days post infection by a rabbit Anti-VHH antibody (Genscript) via flow cytometry (BD FACsCelesta). CAR positive rates and geometric mean expression (mean fluorescent intensity, MFI) were further analyzed by Flowjo 7.6. As shown in FIGS. 2A-2D, DLL3 CARs were successfully expressed in each CAR-T groups with CAR positivity of 55.9%, 17.7% and 28%, respectively. Due to lack of efficient reagents in discriminating the scFv from V_{H}H, the expression of CD56 targeting moieties co-expressed with DLL3 CAR in the DLL3/CD56 Dual-CAR-T cells were not monitored.

### Example 3: Cytotoxicity of Engineered Immune Cells

To assay the cytotoxicity of engineered immune cells, in this case the CAR-T cells of Example 2, towards tumor associated antigen (TAA) expressing tumor cells, an LDH (lactate dehydrogenase) based cytotoxicity assay were performed. Briefly, T cells were cultured with different tumor cell lines (DLL3-positive SHP-77 cells, SHP-77-Luc-DLL3 KO or NK-92 cells) with the E:T ratio of 0.5:1 and 2:1 for 24 hours, respectively.

The SHP-77 cells are DLL3 and CD56 positive. The SHP-77-Luc-DLL3 KO cells were generated by CRISPR-Cas 9-mediated DLL3 knockout in parental SHP-77 cells through GenScript's service. The SHP-77-Luc-DLL3 KO cells are DLL3 negative and CD56 positive. NK-92 cells are DLL3 negative and CD56 positive.

The release of LDH from the dead and dying cells in the supernatant was detected by PHERStar microplate reader according to manufacturer's protocol (Roche, #11644793001). Baseline LDH released from the target cell in the absence of effector cells and effector cell in the absence of target cells were subtracted from the total LDH amount. The target maximum release was obtained by adding Triton-X100 at a final concentration of 1% to target cells in the absence of effector cells at the time when the cytotoxicity assays were initiated. Supernatant from target cells in the absence of effector cells was used for target minimum release. The specific cytotoxicity was calculated by the formula: % Target cell lysis=100×[(OD CAR-T cell+Target cell)−(OD CAR−T cell)−(OD Target cell)+(OD Buffer background)]/(OD Target Maximum release−OD Target Minimum release).

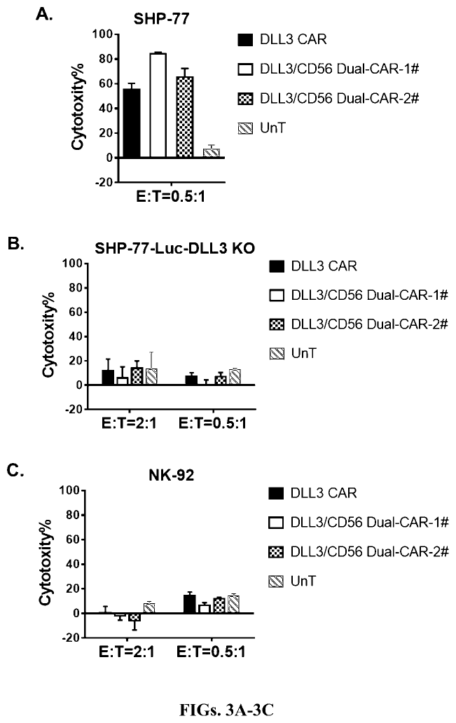

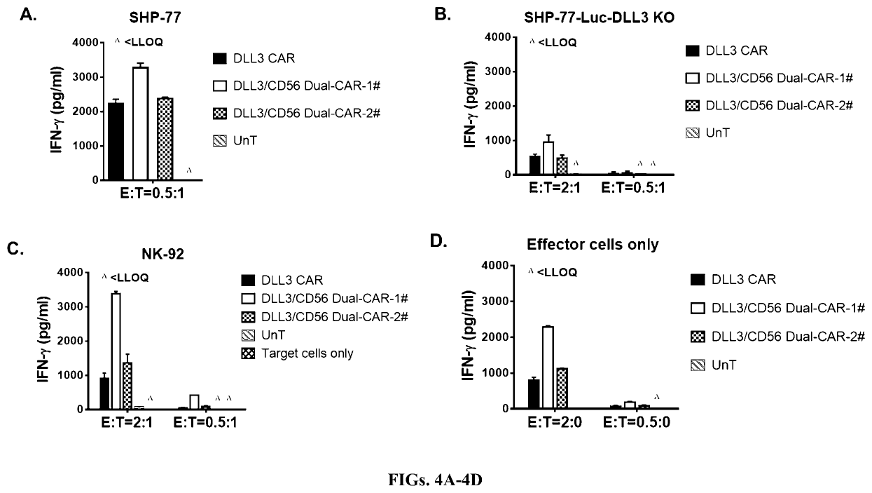

As shown in FIGS. 3A-3C, UnT showed little cytotoxicity against tumor cells. Specific cytotoxicity was observed in CAR-T cell groups against DLL3-positive SHP-77 cells (FIG. 3A). DLL3 CAR cells exhibited approximately 50% cytotoxicity; DLL3/CD56 Dual-CAR cells exhibited about 80% cytotoxicity for DLL3/CD56 Dual-CAR-1 #(60% higher cytotoxicity than DLL3 single CAR cells); and about 65% cytotoxicity for DLL3/CD56 Dual-CAR-2 #(30% higher cytotoxicity than DLL3 single CAR cells). Notably, only little or background cytolysis was observed in CAR-T cells against DLL3 negative and CD56 positive SHP-77-DLL3 knock-out cells (SHP-77-Luc-DLL3 KO) (FIG. 3B) or NK-92 cells (FIG. 3C). The results indicated that functional DLL3-restricted cytolysis in current dual-targeting strategy, can significantly reduce the risk of introducing the 2^{nd} TAA.

### Example 4: IFN-γ Released by Engineered Immune Cells

To further analyze the effect of engineered immune, in this case the engineered T cells of Example 2, against tumor cells, interferon-γ (IFN-γ) release was analyzed in the culture supernatant using the HTRF human IFN gamma kit (Cisbio, #62HIFNGPEH).

As shown in FIGS. 4A-4D, UnT cells showed little secretion of IFN-γ. DLL3 CAR and DLL3/CD56 Dual-CAR T cells secreted various amounts (2200-3300 pg/ml) of IFN-γ in co-culture with DLL3 positive SHP-77 cells (FIG. 4A). Interestingly, in line with the limited cytotoxicity against DLL3-negative and CD56-positive SHP-77-Luc-DLL3 KO cells, only base line IFN-γ release was observed (FIG. 4B), suggesting limited activation of CAR-T cells. Notably, significant higher cytolysis and baseline cytokine expression were observed in DLL3/CD56 Dual-CAR-1 #in comparison to DLL3/CD56 Dual-CAR-2 #(FIGS. 4A-4D), suggesting that wild type CD28 might favor the spontaneous activation of CAR-T cells. Further mutating CD28 reduced the cytokine release to a level that is similar to that of DLL3 CAR, indicating a contribution of CD28 hinge domain and/or transmembrane domains in mediating the baseline activation of CAR-T cells.

### Example 5: Persistent Cytotoxicity Assayed by Repetitive DLL3-Mediated Stimulations

Persistence of CAR-T cells were evaluated in a repetitive tumor challenge assay.

In brief, 1.2×10⁵ CAR-T cells (DLL3 CAR, DLL3/CD56 Dual-CAR-1 #or DLL3/CD56 Dual-CAR-2 #) were co-cultured with 6×10⁵ SHP-77 cells in a 24 well plate, respectively. Three days later, cells were harvested to determine the relative ratios of viable T cells and tumor cells, CAR-T cells were quantified and re-plated with fresh SHP-77 cells at an ET ratio of 1:5 for the next round.

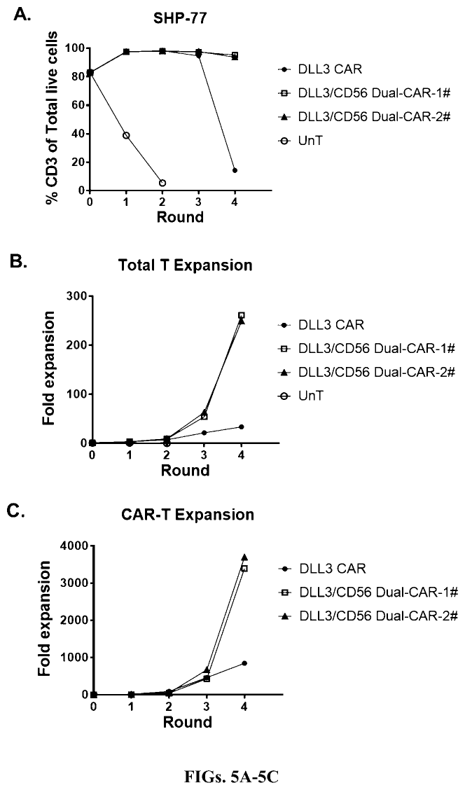

As shown in FIGS. 5A-5C, significant advantages in persistence were observed in DLL3/CD56 Dual-CAR-T cells in comparison to DLL3 CAR-T cells, irrespective of wild type or mutant CD28 hinge domain and transmembrane mediated-downstream signaling upon CD56 engagement. The CAR-T persistence in response to repetitive tumor associated antigen challenges mediated by SHP-77 tumor cells was substantially enhanced (with at least one-round advantage) by introducing another tumor associated antigen CD56 engaging moiety (FIG. 5A). Consequently, the proliferation of DLL3/CD56 Dual-CAR-T cells was strikingly enhanced in comparison to DLL3 CAR-T cells by about 8-folds and 4-folds for total T cells (FIG. 5B) and CAR-positive T cells (FIG. 5C), respectively.

### Example 6: In Vivo Efficacy of DLL3/CD56 Dual-CAR-T Cells

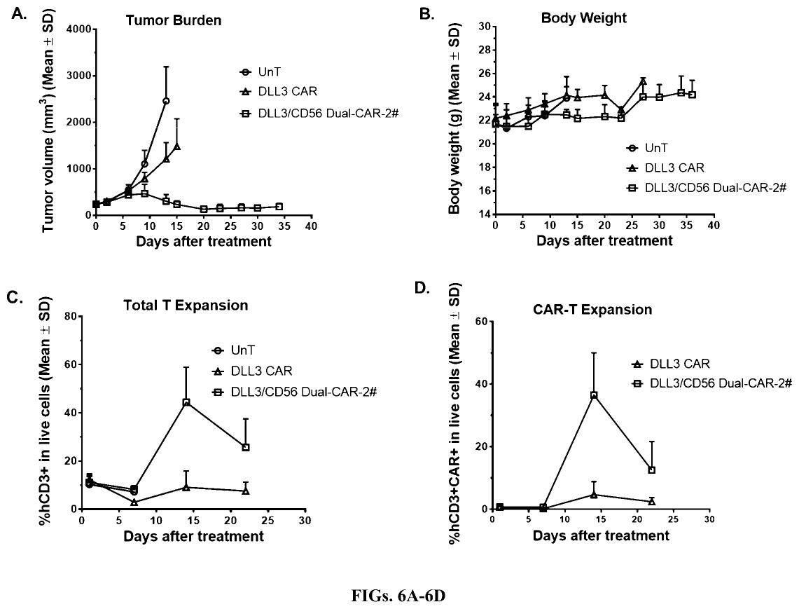

To further demonstrate the antitumor activities of Dual-CAR-T cells in vivo, NCG mice (NOD-PrkdcCd5I12rgCd/NjuCrl) were subcutaneously injected with SCLC NCI-H82 cells. A single dose of un-transduced T cells (8.8×10⁶) or CAR-T cells (1.5×10⁶) was administered intravenously to tumor engrafted mice 10 days after tumor inoculation. Tumor length (L) and width (W) were measured by caliper every 3-4 days after CAR-T cells injection. Tumor volume was estimated using formula: V=(W2×L)/2. Fourteen days after treatment, all the NCG mice had reduced tumor burden in the DLL3 CAR-T treatment group and DLL3/CD56 Dual-CAR-T treatment groups compared with the UnT group (FIG. 6A). Tumor regression was more pronounced in the DLL3/CD56 Dual-CAR-T treatment groups compared with the DLL3 CAR-T treatment group. All DLL3 CAR-T cells treated mice (5/5) suppressed tumor growth but failed to eliminate the xenograft tumor (FIG. 6A). DLL3 CAR-T treated mice were sacrificed starting from day 14 post treatment due to disease progression (FIG. 6A). In comparison, 5/5 (100%) of DLL3/CD56 Dual-CAR-T cells treated mice had durable tumor remission. The data highlighted the superiority of DLL3/CD56 Dual-CAR-T cells in tumor regression and protection of mice from disease progression.

No significant weight loss was observed from NCG mice receiving either DLL3 CAR-T and DLL3/CD56 Dual-CAR-T cells, suggesting that both DLL3 CAR-T and DLL3/CD56 Dual-CAR-T cells were well tolerated by NCG mice (FIG. 6B).

Moreover, the expansion and persistence of CAR-T cells in vivo are also considered as critical predictors of durable clinical tumor regression in subjects with cancer. To understand the basic kinetics of infused CAR-T cells, the percentage of CAR-T cells in peripheral blood of NCG mice was assessed using flow cytometry. As shown in FIGS. 6C-6D, elevated percentage of total T and CAR-T cells in peripheral blood of NCG mice was observed 14 days post treatment. On day 14, 4.67±4.18% of CAR positive T cells were found in the peripheral blood of NCG mice treated with 1.5×10⁶ DLL3 CAR-T cells (FIG. 6D). Percentage of CAR positive T cells in peripheral blood of DLL3/CD56 Dual-CAR-T treated mice (36.55±13.48% on day 14) was at least 9 folds higher than that of DLL3 CAR-T cells (FIG. 6D). The data highlighted the superiority of DLL3/CD56 Dual-CAR-T cells in CAR-T persistence and proliferation.

In line with minimal weight loss, no TAA-independent CAR-T expansion was observed during the study as reflected by constrained proliferation at day 21.

### Example 7: Generation and Evaluation of DLL3/CD56 Dual-CAR-T Cells Expressing an Another DLL3 Bi-VHH CAR

Immune cells (such as T cell) express an another DLL3 bi-VHH CAR (hereinafter named as DLL3 biCAR) comprising two anti-DLL3 VHH domains in the antigen binding domain were also generated. CAR backbone nucleotide sequences encoding DLL3 biCAR, DLL3/CD56 Dual-biCAR-1 #and DLL3/CD56 Dual-biCAR-2 #polypeptides were chemically synthesized and cloned into a pre-modified lentiviral vector (PLVX-EF1A), respectively. The anti-DLL3 VHH domains are different with the anti-DLL3 VHHs disclosed in the Examples above. Briefly, the DLL3 biCAR comprises a polypeptide from the N-terminus to the C-terminus: a CD8α signal peptide (SEQ ID NO: 30), an antigen binding domain having another two anti-DLL3 VHH domains, a CD8α hinge domain (SEQ ID NO: 31), a CD8α transmembrane domain (SEQ ID NO: 34), a 4-1BB co-stimulatory signaling domain (SEQ ID NO: 37), and CD3ζ primary intracellular signaling domain (SEQ ID NO: 54). The DLL3/CD56 Dual-biCAR-1 #and DLL3/CD56 Dual-biCAR-2 #comprise a nucleotide encoding a DLL3 biCAR and a CD56 CAR. The nucleotide encoding the DLL3 biCAR and the nucleotide encoding the CD56 CAR were connected by a nucleotide encoding a 2A linker. The CD56 CAR in the DLL3/CD56 Dual-biCAR-1 #comprises a polypeptide from the N-terminus to the C-terminus: a CD8α signal peptide, an antigen binding domain having an anti-CD56 scFv, a CD28 hinge domain, a CD28 transmembrane domain, and a CD28 co-stimulatory signaling domain, and comprises the amino acid sequence of SEQ ID NO:42. The CD56 CAR in the DLL3/CD56 Dual-biCAR-2 #comprises a polypeptide from the N-terminus to the C-terminus: a CD8α signal peptide, an antigen binding domain having an anti-CD56 scFv, a mutant CD28 hinge domain, a mutant CD28 transmembrane domain, and a CD28 co-stimulatory signaling domain, and comprises the amino acid sequence of SEQ ID NO:42.

CAR positive rates were analyzed by Flowjo v10.9.0. DLL3 biCAR, DLL3/CD56 Dual-biCAR-1 #, and DLL3/CD56 Dual-biCAR-2 #were successfully expressed in each CAR-T groups with CAR positivity of 23.2%, 52.2%, and 57.8%, respectively.

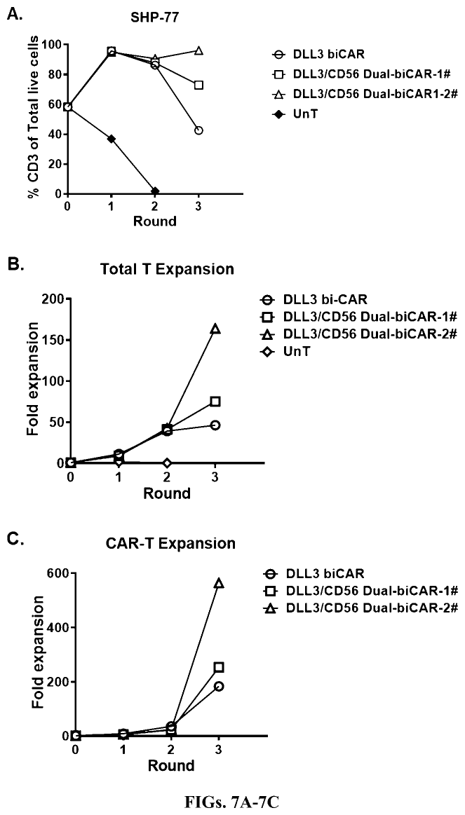

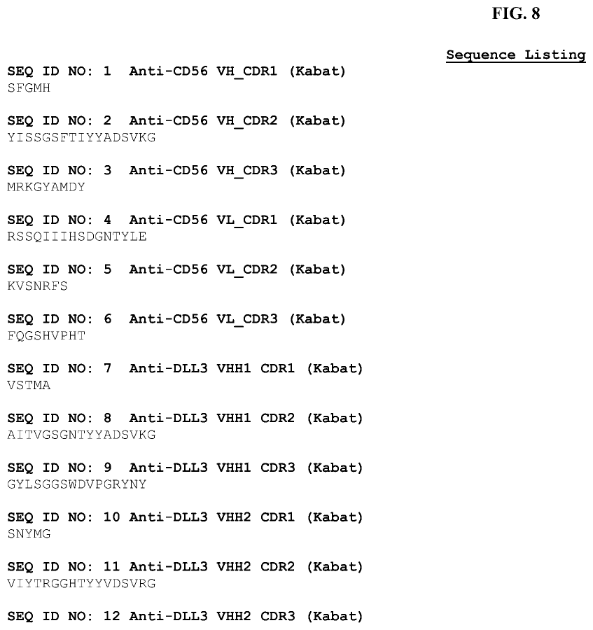

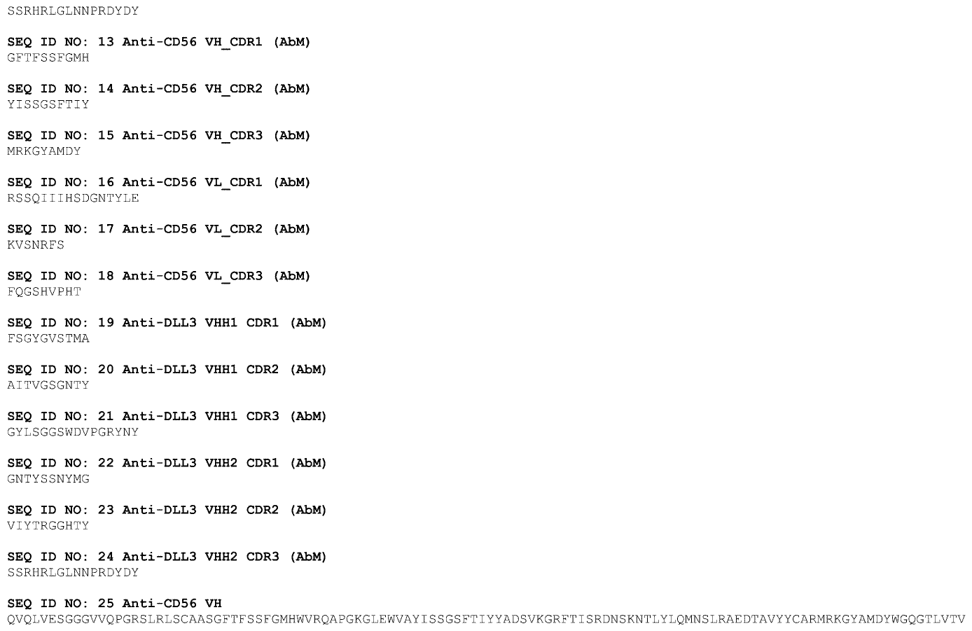

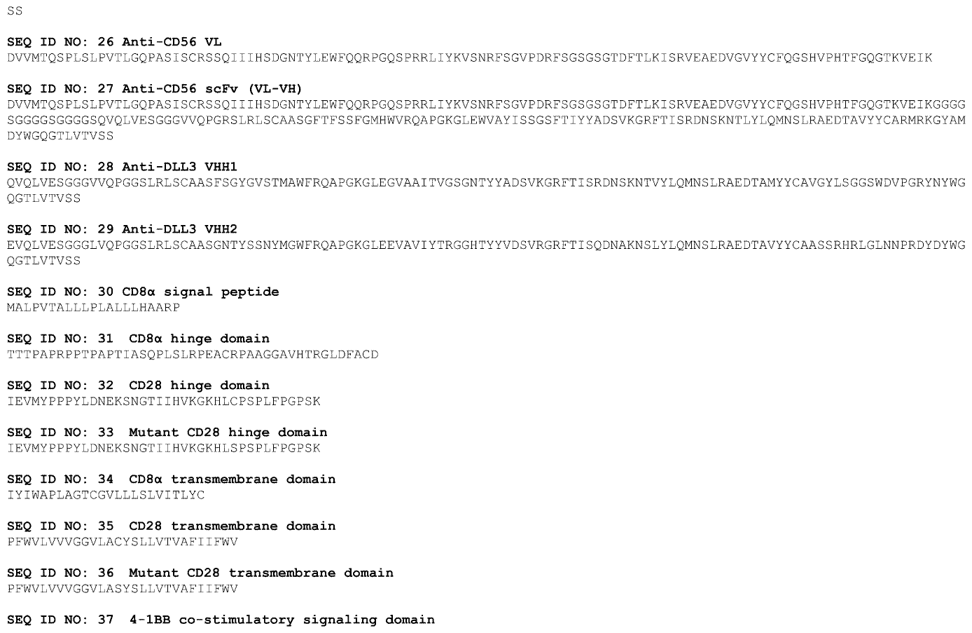

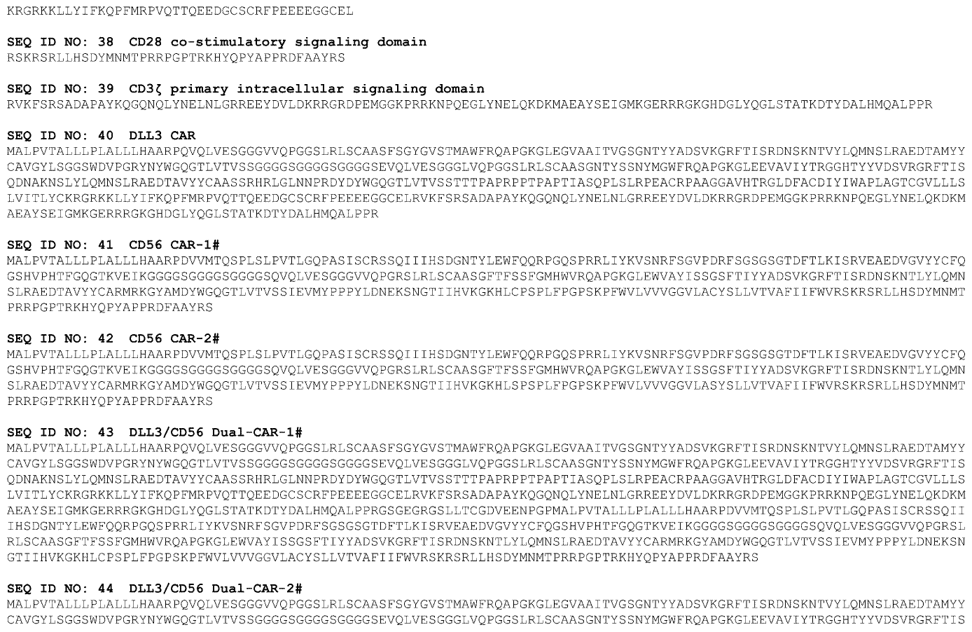

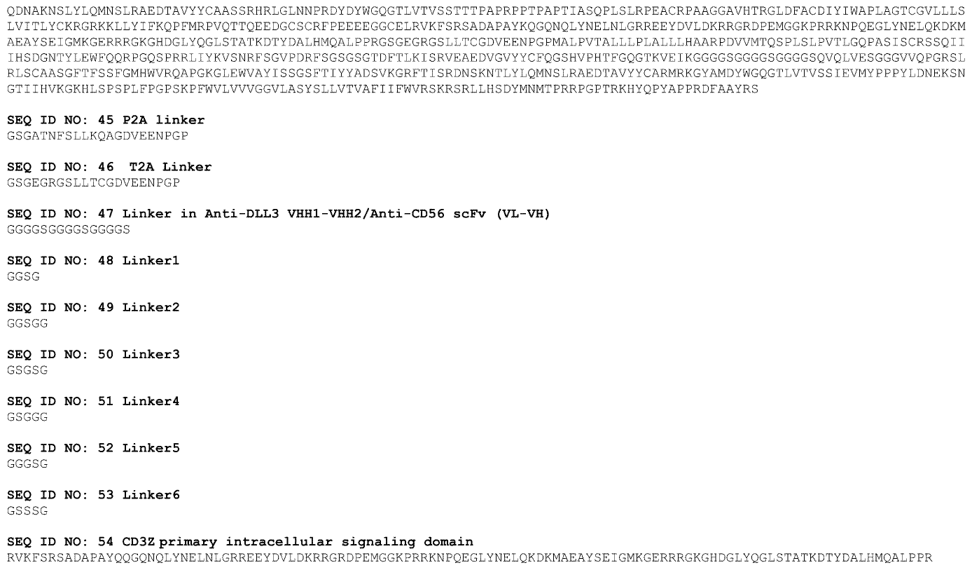

Persistence of CAR-T cells were evaluated in a repetitive tumor challenge assay. In brief, 0.5×10⁵ CAR-T cells (DLL3 biCAR, DLL3/CD56 Dual-biCAR-1 #, and DLL3/CD56 Dual-biCAR-2 #) were co-cultured with 3×10⁵ SHP-77 cells in a 24 well plate, respectively. Three days later, cells were harvested to determine the relative ratios of viable T cells and tumor cells, CAR-T cells were quantified and re-plated with fresh SHP-77 cells at an E:T ratio of 1:5 for the next round. As shown in FIGS. 7A-7C, significant advantages in persistence were observed in DLL3/CD56 Dual-biCAR-1 #, DLL3/CD56 Dual-biCAR-2 #CAR-T cells in comparison to DLL3 biCAR. The CAR-T persistence in response to repetitive tumor associated antigen challenges mediated by SHP-77 tumor cells was substantially enhanced (with at least one-round advantage) by introducing another tumor associated antigen CD56 engaging moiety (FIG. 7A). Consequently, the proliferation of DLL3/CD56 Dual-biCAR-1 #, and DLL3/CD56 Dual-biCAR-2 #CAR-T cells were strikingly enhanced in comparison to DLL3 biCAR-T cells by about 1.5-folds to 3-folds for total T cells (FIG. 7B), and about 1.3-folds to 3 folds for CAR-positive T cells (FIG. 7C).

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. An engineered immune cell comprising a dual chimeric antigen receptor (Dual-CAR) system comprising:
(a) a first chimeric antigen receptor (CAR) comprising:
i) a first antigen binding domain specifically recognizing a first antigen;
ii) a first transmembrane domain; and
iii) a first intracellular signaling domain comprising a co-stimulatory signaling domain of the first intracellular signaling domain; and
(b) a second chimeric antigen receptor (CAR) comprising:
i) a second antigen binding domain specifically recognizing a second antigen;
ii) a second transmembrane domain; and
iii) a second intracellular signaling domain comprising a co-stimulatory signaling domain of the second intracellular signaling domain,
wherein the first intracellular signaling domain further comprises a first primary intracellular signaling domain, and the second intracellular signaling domain does not comprise a primary intracellular signaling domain that is derived from CD3ζ; and
wherein the first antigen and the second antigen are selected from the group consisting of DLL3, CD56, MUCIC, CDH17, GD2, Netrin3, SEZ6, GPC2 and B7-H3.

2. An engineered immune cell comprising a dual chimeric antigen receptor (Dual-CAR) system comprising:
(a) a first chimeric antigen receptor (CAR) comprising:
i) a first antigen binding domain specifically recognizing a first antigen;
ii) a first transmembrane domain; and
iii) a first intracellular signaling domain comprising a co-stimulatory signaling domain of the first intracellular signaling domain; and
(b) a second chimeric antigen receptor (CAR) comprising:
i) a second antigen binding domain specifically recognizing a second antigen;
ii) a second transmembrane domain; and
iii) a second intracellular signaling domain comprising a co-stimulatory signaling domain of the second intracellular signaling domain,
wherein the first intracellular signaling domain further comprises a primary intracellular signaling domain of an immune cell, and the second intracellular signaling domain does not comprise a primary intracellular signaling domain that is derived from CD3ζ; and
wherein each of the first antigen and the second antigen is a neuroendocrine lineage marker.

3. The engineered immune cell of claim 1 or claim 2, wherein the second intracellular signaling domain does not comprise a primary intracellular signaling domain of an immune cell.

4. The engineered immune cell of any one of claims 1-3, wherein the first primary intracellular signaling domain is derived from CD3ζ.

5. The engineered immune cell of any one of claims 1-4, wherein the co-stimulatory signaling domain of the first intracellular signaling domain and/or the co-stimulatory signaling domain of the second intracellular signaling domain is derived from a molecule selected from the group consisting of CD27, CD28, 4-1BB, OX40, CD30, CD40, CD3, LFA-1, ICOS, CD2, CD7, LIGHT, NKG2C, B7-H3, and ligands of CD83.

6. The engineered immune cell of any one of claims 1-5, wherein the co-stimulatory signaling domain of the first intracellular signaling domain comprises a cytoplasmic signaling domain of 4-1BB and the co-stimulatory signaling domain of the second intracellular signaling domain comprises a cytoplasmic signaling domain of CD28.

7. The engineered immune cell of any one of claims 1-6, wherein the first antigen is same with the second antigen, for example wherein the first antigen in DLL3 and the second antigen is DLL3.

8. The engineered immune cell of any one of claims 1-6, wherein the first antigen is different from the second antigen.

9. The engineered immune cell of claim 8, wherein the first antigen is selected from the group consisting of DLL3, GD2, and GPC2.

10. The engineered immune cell of any one of claims 8-9, wherein the second antigen is selected from the group consisting of CD56, MUCIC, CDH17, Netrin3, SEZ6, and B7-H3.

11. The engineered immune cell of any one of claims 1-10, wherein the first and/or the second binding domain comprise a first antigen binding moiety and/or a second antigen binding moiety selected from a Fab, a Fab′, a F(ab′)2, an Fv, a single-chain Fv (scFv), minibody, a diabody, a single-domain antibody (sdAb) or VHH domain.

12. The engineered immune cell of any one of claims 1-11, wherein the second antigen is CD56; and/or wherein the second antigen binding domain comprises an anti-CD56 scFv.

13. The engineered immune cell of any one of claims 1-12, wherein the second antigen is CD56 and the second antigen binding domain comprises an anti-CD56 scFv comprising a VH domain comprising the amino acid sequence of SEQ ID NO: 25; and a VL domain comprising the amino acid sequence of SEQ ID NO: 26.

14. The engineered immune cell of any one of claims 12-13, wherein the anti-CD56 scFv comprises:
(i) a VH domain comprising a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3, wherein the HCDRs are defined according to the Kabat numbering scheme; and
(ii) a VL domain comprising a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, wherein the LCDRs are defined according to the Kabat numbering scheme;
or wherein the anti-CD56 scFv comprises:
(iii) a VH domain comprising a HCDR1 comprising the amino acid sequence of SEQ ID NO: 13, a HCDR2 comprising the amino acid sequence of SEQ ID NO: 14, and a HCDR3 comprising the amino acid sequence of SEQ ID NO: 15, wherein the HCDRs are defined according to the AbM numbering scheme; and
(iv) a VL domain comprising a LCDR1 comprising the amino acid sequence of SEQ ID NO: 16, a LCDR2 comprising the amino acid sequence of SEQ ID NO: 17, and a LCDR3 comprising the amino acid sequence of SEQ ID NO: 18, wherein the LCDRs are defined according to the AbM numbering scheme.

15. The engineered immune cell of any one of claims 1-14, wherein the first antigen is DLL3; and/or wherein the first antigen binding domain comprises one or more anti-DLL3 sdAbs.

16. The engineered immune cell any one of claims 1-15, wherein the first antigen binding domain comprises two anti-DLL3 sdAbs, the two anti-DLL3 sdAbs comprising:
(i) a first anti-DLL3 sdAb comprising a CDR1 comprising the amino acid sequence of SEQ ID NO: 7, a CDR2 comprising the amino acid sequence of SEQ ID NO: 8, and a CDR3 comprising the amino acid sequence of SEQ ID NO: 9, wherein the CDRs are defined according to the Kabat numbering scheme; and
(ii) a second anti-DLL3 sdAb comprising a CDR1 comprising the amino acid sequence of SEQ ID NO: 10, a CDR2 comprising the amino acid sequence of SEQ ID NO: 11, and a CDR3 comprising the amino acid sequence of SEQ ID NO: 12, wherein the CDRs are defined according to the Kabat numbering scheme;
or wherein the two anti-DLL3 sdAbs comprise:
(iii) a first anti-DLL3 sdAb comprising a CDR1 comprising the amino acid sequence of SEQ ID NO: 19, a CDR2 comprising the amino acid sequence of SEQ ID NO: 20, and a CDR3 comprising the amino acid sequence of SEQ ID NO: 21, wherein the CDRs are defined according to the AbM numbering scheme; and
(iv) a second anti-DLL3 sdAb comprising a CDR1 comprising the amino acid sequence of SEQ ID NO: 22, a CDR2 comprising the amino acid sequence of SEQ ID NO: 23, and a CDR3 comprising the amino acid sequence of SEQ ID NO: 24, wherein the CDRs are defined according to the AbM numbering scheme.

17. The engineered immune cell of any one of claims 1-16, wherein the first antigen binding domain comprises a first anti-DLL3 sdAb comprising the amino acid sequence of SEQ ID NO: 28, and a second anti-DLL3 sdAb comprising the amino acid sequence of SEQ ID NO: 29.

18. The engineered immune cell of any one of claims 1-17, wherein the first and/or the second transmembrane domain comprises a transmembrane domain that is derived from a molecule selected from the group consisting of CD8α, CD4, CD28, 4-1BB, CD80, CD86, CD152 and PD1.

19. The engineered immune cell of any of claims 1-18, wherein the first transmembrane domain and/or the second transmembrane domain comprise an amino acid sequence that is at least 95%, 99%, or 100% identical to the amino acid sequence set forth in any one of SEQ ID NOs: 34-36.

20. The engineered immune cell of any one of claims 1-19, wherein the first CAR comprises a hinge domain located between the C-terminus of the first antigen binding domain and the N-terminus of the first transmembrane domain; and wherein the hinge domain of the first chimeric antigen receptor is derived from CD8α or CD28; and the second CAR comprises a hinge domain located between the C-terminus of the second antigen binding domain and the N-terminus of the second transmembrane domain; and wherein the hinge domain of the second chimeric antigen receptor is derived from CD8α or CD28.

21. The engineered immune cell of claim 20, wherein the hinge domain of the first CAR and/or the hinge domain of the second CAR comprises an amino acid sequence that is at least 95%, 99%, or 100% identical to the amino acid sequence set forth in any one of SEQ ID NOs: 31-33.

22. The engineered immune cell of any of claims 20-21, wherein the hinge domain of the second CAR comprises the amino acid sequence of SEQ ID NO: 33 or SEQ ID NO:32, and/or wherein the second transmembrane domain comprises the amino acid sequence of SEQ ID NO: 36 or SEQ ID NO: 35.

23. The engineered immune cell of any one of claims 1-22, wherein each of the first CAR comprises a signal peptide located at the N-terminus of the first antigen binding domain, optionally wherein the signal peptide is derived from CD8α; and/or the second CAR comprises a signal peptide located at the N-terminus of the second antigen binding domain, optionally wherein the signal peptide is derived from CD8α.

24. The engineered immune cell of any one of claims 1-23, wherein the first CAR comprises an amino acid sequence that is at least 95%, 99%, or 100% identical to the amino acid sequence of SEQ ID NO: 40; and/or wherein the second CAR comprises an amino acid sequence that is at least 95%, 99%, or 100% identical to the amino acid sequence of SEQ ID NO: 41 or SEQ ID NO: 42.

25. The engineered immune cell of any one of claims 1-24, comprising a polypeptide comprising the first CAR and/or the second CAR having an amino acid sequence selected from a group consisting of SEQ ID NOs: 40-44.

26. A nucleic acid comprising one or more nucleic acid sequences that encode a first chimeric antigen receptor (CAR) and a second chimeric antigen receptor (CAR), wherein
(a) the first chimeric antigen receptor (CAR) comprising:
i) an antigen binding domain specifically recognizing a first antigen;
ii) a first transmembrane domain; and
iii) a first intracellular signaling domain comprising a co-stimulatory signaling domain of the first intracellular signaling domain; and
(b) the second chimeric antigen receptor (CAR) comprising:
i) a second antigen binding domain specifically recognizing a second antigen;
ii) a second transmembrane domain; and
iii) a second intracellular signaling domain comprising a co-stimulatory signaling domain of the second intracellular signaling domain,
wherein the first intracellular signaling domain further comprises a first primary intracellular signaling domain of an immune cell, and the second intracellular signaling domain does not comprise a primary intracellular signaling domain that is derived from CD3ζ; and
wherein the first antigen and the second antigen are selected from the group consisting of DLL3, CD56, MUCIC, CDH17, GD2, Netrin3, SEZ6, GPC2 and B7-H3.

27. The nucleic acid of claim 26, wherein the second intracellular signaling domain does not comprise a primary intracellular signaling domain of an immune cell.

28. The nucleic acid of claim 26 or claim 27, comprising one or more nucleic acid sequences encoding:
a. the first CAR having an amino acid sequence at least 95%, 99%, or 100% identical to an amino acid of SEQ ID NO: 40; and/or
b. the second CAR having an amino acid sequence at least 95%, 99%, or 100% identical to an amino acid of SEQ ID NO: 41, or SEQ ID NO: 42; or
c. the first CAR and the second CAR having an amino acid sequence at least 95%, 99%, or 100% identical to an amino acid of SEQ ID NO: 43 or SEQ ID NO: 44.

29. The nucleic acid of any one of claims 26-28, comprising a first nucleic acid sequence encoding the first CAR and a second nucleic acid sequence encoding the second CAR, wherein
(i) the first nucleic acid and the second nucleic acid are operably linked to the same promoter;
(ii) the first nucleic acid is upstream of the second nucleic acid or wherein the first nucleic acid is downstream of the second nucleic acid; and/or
(iii) the first nucleic acid and the second nucleic acid are connected via a nucleic acid sequence encoding a linker sequence of P2A or T2A.

30. A single chimeric antigen receptor (CAR) comprising:
(a) an antigen binding domain comprising an anti-CD56 binding moiety specifically recognizing CD56;
(b) a transmembrane domain; and
(c) an intracellular signaling domain,
wherein the anti-CD56 binding moiety comprises a VH domain comprising the amino acid sequence of SEQ ID NO: 25; and a VL domain comprising the amino acid sequence of SEQ ID NO: 26.

31. The single CAR of claim 30, wherein the anti-CD56 binding moiety comprises:
(i) a VH domain comprises a HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3, wherein the HCDRs are defined according to the Kabat numbering scheme; and
(ii) a VL domain comprises a LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6, wherein the LCDRs are defined according to the Kabat numbering scheme;
or:
(iii) a VH domain comprises a HCDR1 comprising the amino acid sequence of SEQ ID NO: 13, a HCDR2 comprising the amino acid sequence of SEQ ID NO: 14, and a HCDR3 comprising the amino acid sequence of SEQ ID NO: 15, wherein the HCDRs are defined according to the AbM numbering scheme; and
(iv) a VL domain comprises a LCDR1 comprising the amino acid sequence of SEQ ID NO: 16, a LCDR2 comprising the amino acid sequence of SEQ ID NO: 17, and a LCDR3 comprising the amino acid sequence of SEQ ID NO: 18, wherein the LCDRs are defined according to the AbM numbering scheme.

32. The single CAR of any one of claims 30-31, wherein the intracellular signaling domain comprises a co-stimulatory signaling domain and does not comprise a primary intracellular signaling domain of an immune cell.

33. The single CAR of any one of claims 30-32, wherein the co-stimulatory signaling domain comprises an amino acid sequence of a cytoplasmic domain that is at least 95% identical to an amino acid sequence of a cytoplasmic domain of CD28 and/or 4-1BB.

34. The single CAR of any one of claims 30-33, wherein the transmembrane domain is derived from a molecule selected from the group consisting of CD8α, CD4, CD28, CD137, CD80, CD86, CD152 and PD1.

35. The single CAR of any one of claims 30-34, wherein the single CAR further comprises a hinge domain derived from CD8α or CD28.

36. The single CAR of any one of claims 30-35, wherein the single CAR further comprises a signal peptide domain derived from CD8α.

37. The single CAR of any one of claims 30-36, wherein the hinge domain is at least 95% identical to the amino acid sequence set forth in any one of SEQ ID NOs: 31-33, and/or the transmembrane domain is at least 95% identical to the amino acid sequence set forth in any one of SEQ ID NOs: 34-36, and/or the signal peptide domain is at least 95% identical to the amino acid sequence of SEQ ID NO: 30.

38. The single CAR of any one of claims 30-37, wherein the single CAR comprises an amino acid sequence that is at least 95%, 99%, or 100% identical to the amino acid sequence set forth in SEQ ID NO: 41 or SEQ ID NO: 42.

39. A nucleic acid comprising one or more nucleic acid sequences that encode the single CAR of any one of claims 30-38.

40. A vector comprising the nucleic acid of claims 26-29, or the nucleic acid of claim 39.

41. Use of the vector of claim 40 to produce an engineered immune cell.

42. An engineered immune cell comprising the single CAR of any one of claims 30-38, or the nucleic acid of claims 26-29, or the nucleic acid of claim 39, or the vector of claim 40.

43. The engineered immune cell of any one of claims 1-25 and 42, wherein the engineered immune cell is selected from the group consisting of T cell, αβT cell, γδT cell, NK cell, peripheral blood mononuclear cell (PBMC), hematopoietic stem cell, pluripotent stem cell, an embryonic stem cell, and a combination thereof.

44. A pharmaceutical composition comprising the engineered immune cell of any one of claims 1-25 and 42-43, and a pharmaceutically acceptable carrier.

45. A method for producing the engineered immune cell of any one of claims 1-25 and 42-43, the method comprising introducing the vector of claim 40 into a cell.

46. A method of treating a subject having cancer or at risk of having cancer, the method comprising: administering the engineered immune cell of any one of claims 1-25, the pharmaceutical composition of claim 44, or the engineered immune cell of claims 42-43 into a subject having cancer or at risk of having cancer.

47. The method of claim 46, wherein the subject has a neuroendocrine tumor (NET).

48. The method of claim 46 or claim 47, wherein the cancer is small cell lung cancer (SCLC), large cell neuroendocrine cancer (LCNC), neuroendocrine prostate cancer (NEPC), pancreatic neuroendocrine tumor (PNET) or gastrointestinal neuroendocrine cancers.


SEQUENCE LISTING (54 sequences)

SEQ ID NO: 1 - protein, 5 aa, synthetic construct
  source 1..5: note = Anti-CD56 VH_CDR1 (Kabat)
SFGMH

SEQ ID NO: 2 - protein, 17 aa, synthetic construct
  source 1..17: note = Anti-CD56 VH_CDR2 (Kabat)
YISSGSFTIYYADSVKG

SEQ ID NO: 3 - protein, 9 aa, synthetic construct
  source 1..9: note = Anti-CD56 VH_CDR3 (Kabat)
MRKGYAMDY

SEQ ID NO: 4 - protein, 16 aa, synthetic construct
  source 1..16: note = Anti-CD56 VL_CDR1 (Kabat)
RSSQIIIHSDGNTYLE

SEQ ID NO: 5 - protein, 7 aa, synthetic construct
  source 1..7: note = Anti-CD56 VL_CDR2 (Kabat)
KVSNRFS

SEQ ID NO: 6 - protein, 9 aa, synthetic construct
  source 1..9: note = Anti-CD56 VL_CDR3 (Kabat)
FQGSHVPHT

SEQ ID NO: 7 - protein, 5 aa, synthetic construct
  source 1..5: note = Anti-DLL3 VHH1 CDR1 (Kabat)
VSTMA

SEQ ID NO: 8 - protein, 17 aa, synthetic construct
  source 1..17: note = Anti-DLL3 VHH1 CDR2 (Kabat)
AITVGSGNTYYADSVKG

SEQ ID NO: 9 - protein, 16 aa, synthetic construct
  source 1..16: note = Anti-DLL3 VHH1 CDR3 (Kabat)
GYLSGGSWDVPGRYNY

SEQ ID NO: 10 - protein, 5 aa, synthetic construct
  source 1..5: note = Anti-DLL3 VHH2 CDR1 (Kabat)
SNYMG

SEQ ID NO: 11 - protein, 17 aa, synthetic construct
  source 1..17: note = Anti-DLL3 VHH2 CDR2 (Kabat)
VIYTRGGHTYYVDSVRG

SEQ ID NO: 12 - protein, 16 aa, synthetic construct
  source 1..16: note = Anti-DLL3 VHH2 CDR3 (Kabat)
SSRHRLGLNNPRDYDY

SEQ ID NO: 13 - protein, 10 aa, synthetic construct
  source 1..10: note = Anti-CD56 VH_CDR1 (AbM)
GFTFSSFGMH

SEQ ID NO: 14 - protein, 10 aa, synthetic construct
  source 1..10: note = Anti-CD56 VH_CDR2 (AbM)
YISSGSFTIY

SEQ ID NO: 15 - protein, 9 aa, synthetic construct
  source 1..9: note = Anti-CD56 VH_CDR3 (AbM)
MRKGYAMDY

SEQ ID NO: 16 - protein, 16 aa, synthetic construct
  source 1..16: note = Anti-CD56 VL_CDR1 (AbM)
RSSQIIIHSDGNTYLE

SEQ ID NO: 17 - protein, 7 aa, synthetic construct
  source 1..7: note = Anti-CD56 VL_CDR2 (AbM)
KVSNRFS

SEQ ID NO: 18 - protein, 9 aa, synthetic construct
  source 1..9: note = Anti-CD56 VL_CDR3 (AbM)
FQGSHVPHT

SEQ ID NO: 19 - protein, 10 aa, synthetic construct
  source 1..10: note = Anti-DLL3 VHH1 CDR1 (AbM)
FSGYGVSTMA

SEQ ID NO: 20 - protein, 10 aa, synthetic construct
  source 1..10: note = Anti-DLL3 VHH1 CDR2 (AbM)
AITVGSGNTY

SEQ ID NO: 21 - protein, 16 aa, synthetic construct
  source 1..16: note = Anti-DLL3 VHH1 CDR3 (AbM)
GYLSGGSWDVPGRYNY

SEQ ID NO: 22 - protein, 10 aa, synthetic construct
  source 1..10: note = Anti-DLL3 VHH2 CDR1 (AbM)
GNTYSSNYMG

SEQ ID NO: 23 - protein, 10 aa, synthetic construct
  source 1..10: note = Anti-DLL3 VHH2 CDR2 (AbM)
VIYTRGGHTY

SEQ ID NO: 24 - protein, 16 aa, synthetic construct
  source 1..16: note = Anti-DLL3 VHH2 CDR3 (AbM)
SSRHRLGLNNPRDYDY

SEQ ID NO: 25 - protein, 118 aa, synthetic construct
  source 1..118: note = Anti-CD56 VH
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSS

SEQ ID NO: 26 - protein, 112 aa, synthetic construct
  source 1..112: note = Anti-CD56 VL
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIK

SEQ ID NO: 27 - protein, 245 aa, synthetic construct
  source 1..245: note = Anti-CD56 scFv (VL-VH)
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKGGGGSGGGGSGGGGSQVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSS

SEQ ID NO: 28 - protein, 125 aa, synthetic construct
  source 1..125: note = Anti-DLL3 VHH1
QVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSS

SEQ ID NO: 29 - protein, 125 aa, synthetic construct
  source 1..125: note = Anti-DLL3 VHH2
EVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSS

SEQ ID NO: 30 - protein, 21 aa, synthetic construct
  source 1..21: note = CD8a signal peptide
MALPVTALLLPLALLLHAARP

SEQ ID NO: 31 - protein, 45 aa, synthetic construct
  source 1..45: note = CD8a hinge domain
TTTPAPRPPTPAPTIASQPLSLRPEACRPAAGGAVHTRGLDFACD

SEQ ID NO: 32 - protein, 38 aa, synthetic construct
  source 1..38: note = CD28 hinge domain
IEVMYPPPYLDNEKSNGTIIHVKGKHLCPSPLFPGPSK

SEQ ID NO: 33 - protein, 38 aa, synthetic construct
  source 1..38: note = Mutant CD28 hinge domain
IEVMYPPPYLDNEKSNGTIIHVKGKHLSPSPLFPGPSK

SEQ ID NO: 34 - protein, 24 aa, synthetic construct
  source 1..24: note = CD8a transmembrane domain
IYIWAPLAGTCGVLLLSLVITLYC

SEQ ID NO: 35 - protein, 28 aa, synthetic construct
  source 1..28: note = CD28 transmembrane domain
PFWVLVVVGGVLACYSLLVTVAFIIFWV

SEQ ID NO: 36 - protein, 28 aa, synthetic construct
  source 1..28: note = Mutant CD28 transmembrane domain
PFWVLVVVGGVLASYSLLVTVAFIIFWV

SEQ ID NO: 37 - protein, 42 aa, synthetic construct
  source 1..42: note = 4-1BB co-stimulatory signaling domain
KRGRKKLLYIFKQPFMRPVQTTQEEDGCSCRFPEEEEGGCEL

SEQ ID NO: 38 - protein, 41 aa, synthetic construct
  source 1..41: note = CD28 co-stimulatory signaling domain
RSKRSRLLHSDYMNMTPRRPGPTRKHYQPYAPPRDFAAYRS

SEQ ID NO: 39 - protein, 112 aa, synthetic construct
  source 1..112: note = CD3Z primary intracellular signaling domain
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 509 aa, synthetic construct
  source 1..509: note = DLL3 CAR
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 373 aa, synthetic construct
  source 1..373: note = CD56 CAR-1#
MALPVTALLLPLALLLHAARPDVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKGGGGSGGGGSGGGGSQVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 42 - protein, 373 aa, synthetic construct
  source 1..373: note = CD56 CAR-2#
MALPVTALLLPLALLLHAARPDVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKGGGGSGGGGSGGGGSQVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSIEVMYPPPYLDNEKSNGTIIHVKGKHLSPSPLFPGPSKPFWVLVVVGGVLASYSLLVTVAFIIFWVRSKRSRLLHSDYMNMTPRRPGPTRKHYQPYAPPRDFAAYRS

SEQ ID NO: 43 - protein, 903 aa, synthetic construct
  source 1..903: note = DLL3/CD56 Dual-CAR-1#
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPDVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKGGGGSGGGGSGGGGSQVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 44 - protein, 903 aa, synthetic construct
  source 1..903: note = DLL3/CD56 Dual-CAR-2#
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPDVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKGGGGSGGGGSGGGGSQVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSIEVMYPPPYLDNEKSNGTIIHVKGKHLSPSPLFPGPSKPFWVLVVVGGVLASYSLLVTVAFIIFWVRSKRSRLLHSDYMNMTPRRPGPTRKHYQPYAPPRDFAAYRS

SEQ ID NO: 45 - protein, 22 aa, synthetic construct
  source 1..22: note = P2A linker
GSGATNFSLLKQAGDVEENPGP

SEQ ID NO: 46 - protein, 21 aa, synthetic construct
  source 1..21: note = T2A Linker
GSGEGRGSLLTCGDVEENPGP

SEQ ID NO: 47 - protein, 15 aa, synthetic construct
  source 1..15: note = Linker in Anti-DLL3 VHH1-VHH2/Anti-CD56 scFv (VL-VH)
GGGGSGGGGSGGGGS

SEQ ID NO: 48 - protein, 4 aa, synthetic construct
  source 1..4: note = Linker1
GGSG

SEQ ID NO: 49 - protein, 5 aa, synthetic construct
  source 1..5: note = Linker2
GGSGG

SEQ ID NO: 50 - protein, 5 aa, synthetic construct
  source 1..5: note = Linker3
GSGSG

SEQ ID NO: 51 - protein, 5 aa, synthetic construct
  source 1..5: note = Linker4
GSGGG

SEQ ID NO: 52 - protein, 5 aa, synthetic construct
  source 1..5: note = Linker5
GGGSG

SEQ ID NO: 53 - protein, 5 aa, synthetic construct
  source 1..5: note = Linker6
GSSSG

SEQ ID NO: 54 - protein, 112 aa, synthetic construct
  source 1..112: note = CD3Z primary intracellular signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR